(19) United States
(12) Patent Application Publication
(10) Pub. No.: US 2025/0354146 A1
(43) Pub. Date: Nov. 20, 2025

(54) MODIFIED ANTISENSE OLIGONUCLEOTIDES TARGETING FOXG1

(71) Applicant: EliGab Tx LLC, Manhasset, NY (US)

(72) Inventor: Scott REICH, Manhasset, NY (US)

(21) Appl. No.: 18/872,678

(22) PCT Filed: Jun. 21, 2023
(86) PCT No.: PCT/US2023/068781

§ 371 Date: Dec. 6, 2024

Related U.S. Application Data
(60) Provisional application No. 63/354,488, filed on Jun. 22, 2022.

(51) Int. Cl. C12N 15/113

(52) U.S. Cl. CPC C12N 15/113; C12N 2310/113; C12N 2310/315; C12N 2310/321; C12N 2310/3231

(57) ABSTRACT

Provided herein are compositions and methods for treating and/or ameliorating FOXG1 syndrome or the symptoms associated therewith. The compositions and methods disclosed herein utilize antisense oligonucleotides that target FOXG1 in order to modulate FOXG1 by, for example, increasing the amount of FOXG1 (e.g., mRNA encoding a FOXG1 protein or FOXG1 protein) in a cell, thereby restoring FOXG1 function.

### RELATED APPLICATIONS

This is a U.S. National Stage Application based on International Application No. PCT/US2023/068781, filed on Jun. 21, 2023, and claims priority to U.S. Provisional Patent Application Ser. No. 63/354,488, filed on Jun. 22, 2022, both of which are incorporated herein by reference in their entireties for all purposes.

### INCORPORATION OF THE SEQUENCE LISTING

This application contains a Sequence Listing, which is hereby incorporated herein by reference in its entirety. The accompanying Sequence Listing, named “062691-503001WO—SEQUENCE LISTING—Jun. 26, 2023”, was created on Jun. 26, 2023 and is 2.7 MB.

### BACKGROUND

FOXG1 syndrome is a rare neurodevelopmental disorder associated with heterozygous variants in the forkhead box G1 (FOXG1) gene and is characterized by impaired neurological development and/or altered brain physiology. Observed phenotypes of FOXG1 syndrome primarily include a particular pattern of structural alterations in the brain resulting from de novo mutations in the FOXG1 gene. Such structural alterations include a thin or underdeveloped corpus callosum that connects between the right and left hemispheres of the brain, reduced sulci and gyri formation on the surface of the brain, and/or a reduced amount of white matter. FOXG1 syndrome affects most aspects of development in children and the main clinical features observed in association with FOXG1 variants comprise impairment of postnatal growth, primary (congenital) or secondary (postnatal) microcephaly, severe intellectual disability with absent speech development, epilepsy, stereotypies and dyskinesia, abnormal sleep patterns, unexplained episodes of crying, gastroesophageal reflux, and recurrent aspiration.

### SUMMARY

Provided herein are compositions and methods for treating and/or ameliorating FOXG1 syndrome or the symptoms associated therewith. The compositions and methods disclosed herein utilize antisense oligonucleotides that target FOXG1 in order to modulate FOXG1 by, for example, increasing the amount of functional FOXG1 protein in a cell, thereby restoring or increasing FOXG1 function. The ability to restore or increase functional FOXG1 in cells provides a foundation for the treatment of FOXG1 syndrome or alleviating symptoms associated therewith.

Provided herein are modified antisense oligonucleotides comprising a sequence complementary to a target nucleic acid sequence of a FOXG1 nucleic acid. In some embodiments, the antisense oligonucleotide comprises a modification within an inter-nucleoside linker or within a nucleoside. In some embodiments, the modification comprises a modified inter-nucleoside linker and a modified nucleoside. In some embodiments, the modified antisense oligonucleotide comprises at least 1 to 10 modified inter-nucleoside linkers. In some embodiments, the modified antisense oligonucleotide comprises at least 10 to 20 modified inter-nucleoside linkers. In some embodiments, the modified antisense oligonucleotide comprises at least 1 to 10 modified nucleosides. In some embodiments, the modified antisense oligonucleotide comprises at least 10 to 20 modified nucleosides. In some embodiments, the modified antisense oligonucleotide comprises at least 10%, at least 25%, at least 50%, at least 75%, at least 80%, or at least 90% modified inter-nucleoside linkers. In some embodiments, the modified antisense oligonucleotide comprises at least 10%, at least 25%, at least 50%, at least 75%, at least 80%, or at least 90% modified nucleosides. In some embodiments, the modified antisense oligonucleotide comprises 16 nucleotides, 17 nucleotides, 18 nucleotides, 19 nucleotides, 20 nucleotides, 21 nucleotides, 22 nucleotides, or 23 nucleotides.

In some embodiments, the FOXG1 nucleic acid comprises a 5′ untranslated region (5′ UTR) and a 3′ untranslated region (3′ UTR), wherein the target sequence is located at the 5′ UTR or the 3′ UTR of the FOXG1 nucleic acid. In some embodiments, the modified antisense oligonucleotide hybridizes to a region of FOXG1 selected from any one of the regions of Table 1 or Table 2. In some embodiments, the target sequence is located within a NM_005249.5_2000-2200_as region of the FOXG1 nucleic acid. In some embodiments, the target sequence is located within a NM_005249.5_2900-3000_as region of the FOXG1 nucleic acid. In some embodiments, the modified antisense oligonucleotide is a single-stranded modified oligonucleotide. In some embodiments, the FOXG1 nucleic acid molecule is a ribonucleic acid (RNA). In some embodiments, the RNA molecule is a messenger RNA (mRNA) molecule.

In some embodiments, the modified antisense oligonucleotide inhibits regulatory elements that reduce translation of the FOXG1 RNA. In some embodiments, the modified antisense oligonucleotide inhibits regulatory elements that reduce stability of the FOXG1 RNA. In some embodiments, the modified antisense oligonucleotide inhibits regulatory elements located within the 3′ UTR of the FOXG1 RNA. In some embodiments, the modified antisense oligonucleotide sterically inhibits (1) miRNA binding and suppression of FOXG1 translation and/or (2) an RNA binding protein from binding to a regulatory sequence of the FOXG1 RNA and destabilizing the FOXG1 RNA. In some embodiments, the modified antisense oligonucleotide inhibits nuclease digestion of the FOXG1 RNA. In some embodiments, the cell is located in a brain of an individual. Further provided are methods of modulating expression of a FOXG1 in a cell, comprising contacting the cell with the modified antisense oligonucleotides described herein. In some embodiments, the cell is located in a brain of an individual. Also provided are methods of treating or ameliorating a FOXG1 disease or disorder in an individual having or at risk of having, the FOXG1 disease or disorder, comprising administering to the individual the modified antisense oligonucleotides described herein, thereby treating or ameliorating a FOXG1 disease in the individual.

In some embodiments, the individual is a human. In some embodiments, the individual comprises a mutated FOXG1 gene. In some embodiments, the individual has a FOXG1 disease or disorder. In some embodiments, the FOXG1 disease or disorder is FOXG1 syndrome. In some embodiments, the FOXG1 nucleic acid is a ribonucleic acid (RNA). In some embodiments, the RNA is a messenger RNA (mRNA). In some embodiments, the modified antisense oligonucleotide inhibits regulatory elements that reduce translation or stability of the FOXG1 RNA, thereby increasing the amount of FOXG1 protein in the cell. In some embodiments, modulating expression comprises increasing expression of a FOXG1 protein in the cell. In some embodiments, modulating expression comprises increasing stability or half-life of the FOXG1 nucleic acid in the cell. In some embodiments, modulating expression comprises increasing translation of a FOXG1 protein in the cell. In some embodiments, the modified antisense oligonucleotide is administered to the individual by intrathecal injection, intracerebroventricular injection, inhalation, parenteral injection or infusion, or orally.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 demonstrates exemplary lengths of modifies ASOs.

FIG. 2 demonstrates exemplary modified inter-nucleoside linker formats of modified ASOs.

FIGS. 3A and 3B demonstrates exemplary modified nucleosides formats of modified ASOs.

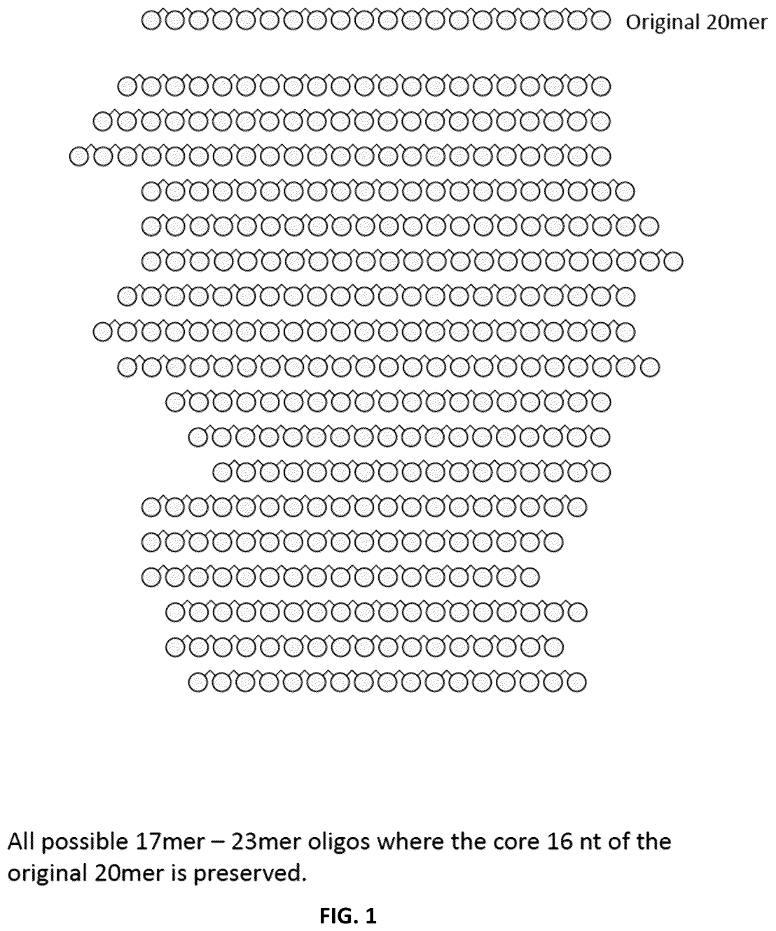

### DETAILED DESCRIPTION

Deletions or mutations in a single allele of the forkhead box G1 (FOXG1) gene cause FOXG1 syndrome. FOXG1 syndrome is a rare disease characterized by developmental delay, severe intellectual disability, epilepsy, absent language, and dyskinesis. Hallmarks of altered brain physiologies associated with FOXG1 syndrome include cortical atrophy and agenesis of the corpus callosum. The FOXG1 gene/protein is a member of the forkhead transcription factor family and is expressed specifically in neural progenitor cells of the forebrain. The FOXG1 gene is composed of one coding exon and notably, the location or type of FOXG1 mutation can be associated with or indicative of clinical severity.

The FOXG1 protein plays an important role in brain development, particularly in a region of the embryonic brain known as the telencephalon. The telencephalon ultimately develops into several critical structures, including the largest part of the brain (i.e., cerebrum), which controls most voluntary activity, language, sensory perception, learning, and memory. A shortage of functional FOXG1 protein, as observed in individuals having mutations or deletions in a single FOXG1 allele (i.e., heterozygous individuals), disrupts normal brain patterning and development.

Accordingly, disclosed herein are compositions and methods useful for increasing an amount of functional FOXG1 (e.g., FOXG1 protein or FOXG1 messenger ribonucleic acid (mRNA)) in a cell having a shortage of functional FOXG1. Such compositions and methods are useful in their application for treating individual having a FOXG1-related disease or disorder wherein the lack or shortage of functional FOXG1 protein can be remedied. In order to achieve an increase of FOXG1 expression in cells or in an individual, antisense oligonucleotides targeting FOXG1 are used.

### Modified ASOs

Antisense oligonucleotides (ASOs) are small (˜18-30 nucleotides), synthetic, single-stranded nucleic acid polymers that can be employed to modulate gene expression by various mechanisms. Antisense oligonucleotides (ASOs) can be subdivided into two major categories: RNase H competent and steric block. For RNase H competent antisense oligonucleotides, the endogenous RNase H enzyme recognizes RNA-DNA heteroduplex substrates that are formed when antisense oligonucleotides bind to their cognate mRNA transcripts to catalyze the degradation of RNA. Steric block oligonucleotides are antisense oligonucleotides (ASOs) that are designed to bind to target transcripts with high affinity but do not induce target transcript degradation.

Steric block antisense oligonucleotides (ASOs) can be designed to inhibit translation inhibition, interfere with upstream open reading frames that negatively regulate translation in order to activate protein expression, inhibit RNA degradation, inhibit miRNA suppression, and influence polyadenylation signals to increase transcript stability. Accordingly, provided herein are steric block antisense oligonucleotides (ASOs) useful for modulating the expression and/or amount of functional FOXG1 (i.e., functional FOXG1) in a cell (e.g., mRNA encoding a functional FOXG1 protein or a FOXG1 protein). Specifically, the antisense oligonucleotides (ASOs) are useful for increasing the expression and/or amount of FOXG1 (i.e., functional FOXG1) in a cell (e.g., mRNA encoding a functional FOXG1 protein or a functional FOXG1 protein). The antisense oligonucleotides (ASOs) disclosed herein achieve this effect by targeting a FOXG1 nucleic acid encoding a functional FOXG1 protein and inhibiting translation inhibition, interfering with upstream open reading frames (uORFs), inhibiting RNA degradation, inhibiting miRNA suppression of expression, and/or increasing RNA stability to ultimately increase the number of RNA transcripts encoding FOXG1 and/or protein expression of a FOXG1 (i.e., functional FOXG1) protein.

In order to achieve effective targeting of a FOXG1 RNA (e.g., messenger RNA), the antisense oligonucleotides disclosed herein (ASOs) comprise a sequence complementary to a sequence of the FOXG1 RNA, wherein the complementary sequence binds and/or hybridizes to a sequence of the FOXG1 RNA. Accordingly, disclosed herein are antisense oligonucleotides (ASOs) comprising a sequence complementary to a target nucleic acid sequence of a FOXG1 nucleic acid (e.g., a FOXG1 mRNA). Generally, mRNA transcripts comprise a 5′ untranslated region (5′ UTR) and a 3′ untranslated region (3′ UTR). The antisense oligonucleotides (ASOs) disclosed herein target the 5′ UTR or the 3′ UTR of a FOXG1 mRNA transcript. In order to achieve targeting of the 5′ UTR or 3′ UTR, the antisense oligonucleotide (ASOs) comprise a sequence complementary to a target sequence located at the 5′ UTR or the 3′ UTR of the FOXG1 mRNA. In some embodiments, the target sequence is located at or within the 5′ UTR. In some embodiments, the target sequence is located at or within the 3′ UTR. In certain embodiments, the antisense oligonucleotide targeting the 3′ UTR comprises a nucleobase sequence complementary to a sequence within the nucleotides contained in the FOXG1 nucleic acid regions specified by the 2000-2200 or 2900-3000 coordinates of the NM_005249.5 mRNA entry in RefSeq. In some embodiments, the antisense oligonucleotides are included in an ASO composition comprising more than one ASO. In certain embodiments, the ASO composition comprises 2, 3, 4, 5, or more ASOs. Such ASO compositions are suitable for use in the methods described herein. TABLES 2-3 disclose regions of the FOXG1 mRNA associated with an increase in FOXG1 expression when targeted by antisense oligonucleotides (ASOs). In some embodiments, the antisense oligonucleotides (ASOs) disclosed herein, targeting the 5′ UTR or 3′ UTR, increase the amount of FOXG1 protein and/or mRNA transcripts in a cell and/or individual. In certain embodiments, targeting a FOXG1 nucleic acid encoding a functional FOXG1 protein inhibits translation inhibition, interferes with upstream open reading frames (uORFs), inhibits RNA degradation, and/or increases RNA stability to ultimately increase protein expression of a functional FOXG1 protein.

In some embodiments, the modified antisense oligonucleotides (ASOs) described herein (e.g., hybridizing to the regions of FOXG1 described in TABLES 2-3) comprise at least 15, at least 16, at least 17, at least 18, at least 19, at least 20, at least 21, at least 22, at least 23, at least 24, or at least 25 nucleotides. In some embodiments, the modified antisense oligonucleotides (ASOs) described herein (e.g., hybridizing to the regions of FOXG1 described in TABLES 2-3) comprise 17 to 23 nucleotides.

In some embodiments, the modified antisense oligonucleotides (ASOs) comprise 15 nucleotides to 25 nucleotides. In some embodiments, the modified antisense oligonucleotides (ASOs) comprise 15 nucleotides to 16 nucleotides, 15 nucleotides to 17 nucleotides, 15 nucleotides to 18 nucleotides, 15 nucleotides to 19 nucleotides, 15 nucleotides to 20 nucleotides, 15 nucleotides to 21 nucleotides, 15 nucleotides to 22 nucleotides, 15 nucleotides to 23 nucleotides, 15 nucleotides to 24 nucleotides, 15 nucleotides to 25 nucleotides, 16 nucleotides to 17 nucleotides, 16 nucleotides to 18 nucleotides, 16 nucleotides to 19 nucleotides, 16 nucleotides to 20 nucleotides, 16 nucleotides to 21 nucleotides, 16 nucleotides to 22 nucleotides, 16 nucleotides to 23 nucleotides, 16 nucleotides to 24 nucleotides, 16 nucleotides to 25 nucleotides, 17 nucleotides to 18 nucleotides, 17 nucleotides to 19 nucleotides, 17 nucleotides to 20 nucleotides, 17 nucleotides to 21 nucleotides, 17 nucleotides to 22 nucleotides, 17 nucleotides to 23 nucleotides, 17 nucleotides to 24 nucleotides, 17 nucleotides to 25 nucleotides, 18 nucleotides to 19 nucleotides, 18 nucleotides to 20 nucleotides, 18 nucleotides to 21 nucleotides, 18 nucleotides to 22 nucleotides, 18 nucleotides to 23 nucleotides, 18 nucleotides to 24 nucleotides, 18 nucleotides to 25 nucleotides, 19 nucleotides to 20 nucleotides, 19 nucleotides to 21 nucleotides, 19 nucleotides to 22 nucleotides, 19 nucleotides to 23 nucleotides, 19 nucleotides to 24 nucleotides, 19 nucleotides to 25 nucleotides, 20 nucleotides to 21 nucleotides, 20 nucleotides to 22 nucleotides, 20 nucleotides to 23 nucleotides, 20 nucleotides to 24 nucleotides, 20 nucleotides to 25 nucleotides, 21 nucleotides to 22 nucleotides, 21 nucleotides to 23 nucleotides, 21 nucleotides to 24 nucleotides, 21 nucleotides to 25 nucleotides, 22 nucleotides to 23 nucleotides, 22 nucleotides to 24 nucleotides, 22 nucleotides to 25 nucleotides, 23 nucleotides to 24 nucleotides, 23 nucleotides to 25 nucleotides, or 24 nucleotides to 25 nucleotides. In some embodiments, the modified antisense oligonucleotides (ASOs) comprise 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides. In some embodiments, the modified antisense oligonucleotides (ASOs) comprise at least 15 nucleotides, 16 nucleotides, 17 nucleotides, 18 nucleotides, 19 nucleotides, 20 nucleotides, 21 nucleotides, 22 nucleotides, 23 nucleotides, or 24 nucleotides. In some embodiments, the modified antisense oligonucleotides (ASOs) comprise at most 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides.

In order to improve the pharmacodynamic, pharmacokinetic, and biodistribution properties of antisense oligonucleotides (ASOs), the antisense oligonucleotides can be designed and engineered to comprise one or more chemical modifications (e.g., a modified inter-nucleoside linker, a modified nucleoside, or a combination thereof). Accordingly, in some embodiments, the antisense oligonucleotide is a modified oligonucleotide. In some embodiments, the antisense oligonucleotide comprises one or more modifications. In certain embodiments, the modification comprises a modified inter-nucleoside linker, a modified nucleoside, or a combination thereof.

### Modified Inter-Nucleoside Linkers

Modification of the inter-nucleoside linker (i.e., backbone) can be utilized to increase pharmacodynamic, pharmacokinetic, and biodistribution properties. For example, inter-nucleoside linker modifications prevent or reduce degradation by cellular nucleases, thus increasing the pharmacokinetics and bioavailability of the modified antisense oligonucleotide. Generally, a modified inter-nucleoside linker includes any linker other than phosphodiester (PO) liners, that covalently couples two nucleosides together. In some embodiments, the modified inter-nucleoside linker increases the nuclease resistance of the modified antisense oligonucleotide compared to a phosphodiester linker. For naturally occurring antisense oligonucleotides, the inter-nucleoside linker includes phosphate groups creating a phosphodiester bond between adjacent nucleosides. Modified inter-nucleoside linkers are particularly useful in stabilizing antisense oligonucleotides for in vivo use and may serve to protect against nuclease cleavage.

In some embodiments, the modified antisense oligonucleotide comprises one or more inter-nucleoside linkers modified from the natural phosphodiester to a linker that is for example more resistant to nuclease attack. In some embodiments, all of the inter-nucleoside linkers of the modified antisense oligonucleotide, or contiguous nucleotide sequence thereof, are modified. In some embodiments, all of the inter-nucleoside linkers of the modified antisense oligonucleotide, or contiguous nucleotide sequence thereof, are nuclease-resistant inter-nucleoside linkers. In some embodiments, the inter-nucleoside linkage comprises sulfur(S), such as a phosphorothioate inter-nucleoside linkage.

Phosphorothioate (PS) inter-nucleoside linkers are particularly useful due to nuclease resistance and improved pharmacokinetics. In some embodiments, one or more of the inter-nucleoside linkers of the modified antisense oligonucleotide, or contiguous nucleotide sequence thereof, comprise a phosphorothioate inter-nucleoside linker. In some embodiments, all of the inter-nucleoside linkers of the modified antisense oligonucleotide, or contiguous nucleotide sequence thereof, comprise a phosphorothioate inter-nucleoside linker.

Representative phosphorus containing internucleoside linkages further include, but are limited to, phosphodiesters, phosphotriesters, methylphosphonates, phosphoramidate, and phosphorothioates. Methods of preparation of phosphorous-containing and non-phosphorous-containing linkages are well known. Generally, the two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH2-N(CH3)-O—CH2-), thiodiester, thionocarbamate (—O—C(═O)(NH)—S—); siloxane (—O—SiH2-O—); and N,N′-dimethylhydrazine (—CH2-N(CH3)-N(CH3)-). Additional examples of inter-nucleoside linkages include, without limitation, phosphotriesters, methylphosphonates, MMI (31-CH2-N(CH3)-O-5′), amide-3 (31-CH2-C(═O)—N(H)-5′), amide-4 (31-CH2-N(H)—C(═O)-5′), formacetal (31-O—CH2-O-5), methoxypropyl, and thioformacetal (31-S-CH2-O-5′). Further inter-nucleoside linkages include nonionic linkages comprising siloxane (dialkylsiloxane), carboxylate ester, carboxamide, sulfide, sulfonate ester, and amides. Modified inter-nucleoside linkages, compared to naturally occurring phosphate linkages, can be used to alter, typically increase, nuclease resistance of the oligonucleotide.

In some embodiments, the modified antisense oligonucleotides (ASOs) comprise one or more modified inter-nucleoside linkers. In certain embodiments, the modified antisense oligonucleotides (ASOs) comprise 1 modified inter-nucleoside linker to 24 modified inter-nucleoside linkers. In certain embodiments, the modified antisense oligonucleotides (ASOs) comprise 1 modified inter-nucleoside linker to 2 modified inter-nucleoside linkers, 1 modified inter-nucleoside linker to 3 modified inter-nucleoside linkers, 1 modified inter-nucleoside linker to 4 modified inter-nucleoside linkers, 1 modified inter-nucleoside linker to 5 modified inter-nucleoside linkers, 1 modified inter-nucleoside linker to 10 modified inter-nucleoside linkers, 1 modified inter-nucleoside linker to 12 modified inter-nucleoside linkers, 1 modified inter-nucleoside linker to 15 modified inter-nucleoside linkers, 1 modified inter-nucleoside linker to 18 modified inter-nucleoside linkers, 1 modified inter-nucleoside linker to 20 modified inter-nucleoside linkers, 1 modified inter-nucleoside linker to 22 modified inter-nucleoside linkers, 1 modified inter-nucleoside linker to 24 modified inter-nucleoside linkers, 2 modified inter-nucleoside linkers to 3 modified inter-nucleoside linkers, 2 modified inter-nucleoside linkers to 4 modified inter-nucleoside linkers, 2 modified inter-nucleoside linkers to 5 modified inter-nucleoside linkers, 2 modified inter-nucleoside linkers to 10 modified inter-nucleoside linkers, 2 modified inter-nucleoside linkers to 12 modified inter-nucleoside linkers, 2 modified inter-nucleoside linkers to 15 modified inter-nucleoside linkers, 2 modified inter-nucleoside linkers to 18 modified inter-nucleoside linkers, 2 modified inter-nucleoside linkers to 20 modified inter-nucleoside linkers, 2 modified inter-nucleoside linkers to 22 modified inter-nucleoside linkers, 2 modified inter-nucleoside linkers to 24 modified inter-nucleoside linkers, 3 modified inter-nucleoside linkers to 4 modified inter-nucleoside linkers, 3 modified inter-nucleoside linkers to 5 modified inter-nucleoside linkers, 3 modified inter-nucleoside linkers to 10 modified inter-nucleoside linkers, 3 modified inter-nucleoside linkers to 12 modified inter-nucleoside linkers, 3 modified inter-nucleoside linkers to 15 modified inter-nucleoside linkers, 3 modified inter-nucleoside linkers to 18 modified inter-nucleoside linkers, 3 modified inter-nucleoside linkers to 20 modified inter-nucleoside linkers, 3 modified inter-nucleoside linkers to 22 modified inter-nucleoside linkers, 3 modified inter-nucleoside linkers to 24 modified inter-nucleoside linkers, 4 modified inter-nucleoside linkers to 5 modified inter-nucleoside linkers, 4 modified inter-nucleoside linkers to 10 modified inter-nucleoside linkers, 4 modified inter-nucleoside linkers to 12 modified inter-nucleoside linkers, 4 modified inter-nucleoside linkers to 15 modified inter-nucleoside linkers, 4 modified inter-nucleoside linkers to 18 modified inter-nucleoside linkers, 4 modified inter-nucleoside linkers to 20 modified inter-nucleoside linkers, 4 modified inter-nucleoside linkers to 22 modified inter-nucleoside linkers, 4 modified inter-nucleoside linkers to 24 modified inter-nucleoside linkers, 5 modified inter-nucleoside linkers to 10 modified inter-nucleoside linkers, 5 modified inter-nucleoside linkers to 12 modified inter-nucleoside linkers, 5 modified inter-nucleoside linkers to 15 modified inter-nucleoside linkers, 5 modified inter-nucleoside linkers to 18 modified inter-nucleoside linkers, 5 modified inter-nucleoside linkers to 20 modified inter-nucleoside linkers, 5 modified inter-nucleoside linkers to 22 modified inter-nucleoside linkers, 5 modified inter-nucleoside linkers to 24 modified inter-nucleoside linkers, 10 modified inter-nucleoside linkers to 12 modified inter-nucleoside linkers, 10 modified inter-nucleoside linkers to 15 modified inter-nucleoside linkers, 10 modified inter-nucleoside linkers to 18 modified inter-nucleoside linkers, 10 modified inter-nucleoside linkers to 20 modified inter-nucleoside linkers, 10 modified inter-nucleoside linkers to 22 modified inter-nucleoside linkers, 10 modified inter-nucleoside linkers to 24 modified inter-nucleoside linkers, 12 modified inter-nucleoside linkers to 15 modified inter-nucleoside linkers, 12 modified inter-nucleoside linkers to 18 modified inter-nucleoside linkers, 12 modified inter-nucleoside linkers to 20 modified inter-nucleoside linkers, 12 modified inter-nucleoside linkers to 22 modified inter-nucleoside linkers, 12 modified inter-nucleoside linkers to 24 modified inter-nucleoside linkers, 15 modified inter-nucleoside linkers to 18 modified inter-nucleoside linkers, 15 modified inter-nucleoside linkers to 20 modified inter-nucleoside linkers, 15 modified inter-nucleoside linkers to 22 modified inter-nucleoside linkers, 15 modified inter-nucleoside linkers to 24 modified inter-nucleoside linkers, 18 modified inter-nucleoside linkers to 20 modified inter-nucleoside linkers, 18 modified inter-nucleoside linkers to 22 modified inter-nucleoside linkers, 18 modified inter-nucleoside linkers to 24 modified inter-nucleoside linkers, 20 modified inter-nucleoside linkers to 22 modified inter-nucleoside linkers, 20 modified inter-nucleoside linkers to 24 modified inter-nucleoside linkers, or 22 modified inter-nucleoside linkers to 24 modified inter-nucleoside linkers. In certain embodiments, the modified antisense oligonucleotides (ASOs) comprise 1 modified inter-nucleoside linker, 2 modified inter-nucleoside linkers, 3 modified inter-nucleoside linkers, 4 modified inter-nucleoside linkers, 5 modified inter-nucleoside linkers, 10 modified inter-nucleoside linkers, 12 modified inter-nucleoside linkers, 15 modified inter-nucleoside linkers, 18 modified inter-nucleoside linkers, 20 modified inter-nucleoside linkers, 22 modified inter-nucleoside linkers, or 24 modified inter-nucleoside linkers. In certain embodiments, the modified antisense oligonucleotides (ASOs) comprise at least 1 modified inter-nucleoside linker, 2 modified inter-nucleoside linkers, 3 modified inter-nucleoside linkers, 4 modified inter-nucleoside linkers, 5 modified inter-nucleoside linkers, 10 modified inter-nucleoside linkers, 12 modified inter-nucleoside linkers, 15 modified inter-nucleoside linkers, 18 modified inter-nucleoside linkers, 20 modified inter-nucleoside linkers, or 22 modified inter-nucleoside linkers. In certain embodiments, the modified antisense oligonucleotides (ASOs) comprise at most 2 modified inter-nucleoside linkers, 3 modified inter-nucleoside linkers, 4 modified inter-nucleoside linkers, 5 modified inter-nucleoside linkers, 10 modified inter-nucleoside linkers, 12 modified inter-nucleoside linkers, 15 modified inter-nucleoside linkers, 18 modified inter-nucleoside linkers, 20 modified inter-nucleoside linkers, 22 modified inter-nucleoside linkers, or 24 modified inter-nucleoside linkers.

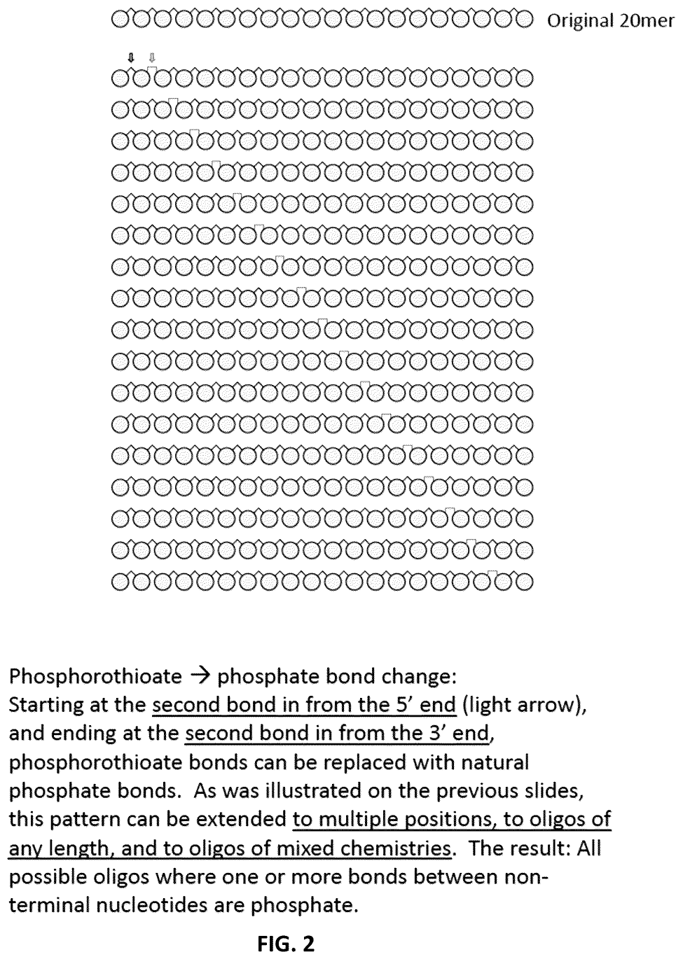

FIG. 2 demonstrates exemplary nucleotide positions (circles) that can comprise the modified inter-nucleoside linker(s). In certain embodiments, the modified antisense oligonucleotides (ASOs) comprise at least 10% modified inter-nucleoside linkers (e.g., 90% of inter-nucleoside linkers are PO phosphate bonds) to at least 100% modified inter-nucleoside linkers. In certain embodiments, the modified antisense oligonucleotides (ASOs) comprise 10% modified inter-nucleoside linkers to 20% modified inter-nucleoside linkers, 10% modified inter-nucleoside linkers to 30% modified inter-nucleoside linkers, 10% modified inter-nucleoside linkers to 40% modified inter-nucleoside linkers, 10% modified inter-nucleoside linkers to 50% modified inter-nucleoside linkers, 10% modified inter-nucleoside linkers to 60% modified inter-nucleoside linkers, 10% modified inter-nucleoside linkers to 70% modified inter-nucleoside linkers, 10% modified inter-nucleoside linkers to 80% modified inter-nucleoside linkers, 10% modified inter-nucleoside linkers to 90% modified inter-nucleoside linkers, 10% modified inter-nucleoside linkers to 100% modified inter-nucleoside linkers, 20% modified inter-nucleoside linkers to 30% modified inter-nucleoside linkers, 20% modified inter-nucleoside linkers to 40% modified inter-nucleoside linkers, 20% modified inter-nucleoside linkers to 50% modified inter-nucleoside linkers, 20% modified inter-nucleoside linkers to 60% modified inter-nucleoside linkers, 20% modified inter-nucleoside linkers to 70% modified inter-nucleoside linkers, 20% modified inter-nucleoside linkers to 80% modified inter-nucleoside linkers, 20% modified inter-nucleoside linkers to 90% modified inter-nucleoside linkers, 20% modified inter-nucleoside linkers to 100% modified inter-nucleoside linkers, 30% modified inter-nucleoside linkers to 40% modified inter-nucleoside linkers, 30% modified inter-nucleoside linkers to 50% modified inter-nucleoside linkers, 30% modified inter-nucleoside linkers to 60% modified inter-nucleoside linkers, 30% modified inter-nucleoside linkers to 70% modified inter-nucleoside linkers, 30% modified inter-nucleoside linkers to 80% modified inter-nucleoside linkers, 30% modified inter-nucleoside linkers to 90% modified inter-nucleoside linkers, 30% modified inter-nucleoside linkers to 100% modified inter-nucleoside linkers, 40% modified inter-nucleoside linkers to 50% modified inter-nucleoside linkers, 40% modified inter-nucleoside linkers to 60% modified inter-nucleoside linkers, 40% modified inter-nucleoside linkers to 70% modified inter-nucleoside linkers, 40% modified inter-nucleoside linkers to 80% modified inter-nucleoside linkers, 40% modified inter-nucleoside linkers to 90% modified inter-nucleoside linkers, 40% modified inter-nucleoside linkers to 100% modified inter-nucleoside linkers, 50% modified inter-nucleoside linkers to 60% modified inter-nucleoside linkers, 50% modified inter-nucleoside linkers to 70% modified inter-nucleoside linkers, 50% modified inter-nucleoside linkers to 80% modified inter-nucleoside linkers, 50% modified inter-nucleoside linkers to 90% modified inter-nucleoside linkers, 50% modified inter-nucleoside linkers to 100% modified inter-nucleoside linkers, 60% modified inter-nucleoside linkers to 70% modified inter-nucleoside linkers, 60% modified inter-nucleoside linkers to 80% modified inter-nucleoside linkers, 60% modified inter-nucleoside linkers to 90% modified inter-nucleoside linkers, 60% modified inter-nucleoside linkers to 100% modified inter-nucleoside linkers, 70% modified inter-nucleoside linkers to 80% modified inter-nucleoside linkers, 70% modified inter-nucleoside linkers to 90% modified inter-nucleoside linkers, 70% modified inter-nucleoside linkers to 100% modified inter-nucleoside linkers, 80% modified inter-nucleoside linkers to 90% modified inter-nucleoside linkers, 80% modified inter-nucleoside linkers to 100% modified inter-nucleoside linkers, or 90% modified inter-nucleoside linkers to 100% modified inter-nucleoside linkers. In certain embodiments, the modified antisense oligonucleotides (ASOs) comprise 10% modified inter-nucleoside linkers, 20% modified inter-nucleoside linkers, 30% modified inter-nucleoside linkers, 40% modified inter-nucleoside linkers, 50% modified inter-nucleoside linkers, 60% modified inter-nucleoside linkers, 70% modified inter-nucleoside linkers, 80% modified inter-nucleoside linkers, 90% modified inter-nucleoside linkers, or 100% modified inter-nucleoside linkers. In certain embodiments, the modified antisense oligonucleotides (ASOs) comprise at least 10% modified inter-nucleoside linkers, 20% modified inter-nucleoside linkers, 30% modified inter-nucleoside linkers, 40% modified inter-nucleoside linkers, 50% modified inter-nucleoside linkers, 60% modified inter-nucleoside linkers, 70% modified inter-nucleoside linkers, 80% modified inter-nucleoside linkers, or 90% modified inter-nucleoside linkers. In certain embodiments, the modified antisense oligonucleotides (ASOs) comprise at most 20% modified inter-nucleoside linkers, 30% modified inter-nucleoside linkers, 40% modified inter-nucleoside linkers, 50% modified inter-nucleoside linkers, 60% modified inter-nucleoside linkers, 70% modified inter-nucleoside linkers, 80% modified inter-nucleoside linkers, 90% modified inter-nucleoside linkers, or 100% modified inter-nucleoside linkers.

The modified inter-nucleoside linkers described herein can be employed across modified antisense oligonucleotides (ASOs) of the varying lengths described herein (e.g., 17-23 mers) and/or in combination with any number of modified nucleosides described herein.

### Modified Nucleosides

Modifications to the ribose sugar or nucleobase can also be utilized to increase pharmacodynamic, pharmacokinetic, and biodistribution properties. Similar to modifications of the inter-nucleoside linker, nucleoside modifications prevent or reduce degradation by cellular nucleases, thus increasing the pharmacokinetics and bioavailability of the modified antisense oligonucleotide. Generally, a modified nucleoside includes the introduction of one or more modifications of the sugar moiety or the nucleobase moiety.

The modified antisense oligonucleotides, as described, can comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or nuclease resistance. Such modifications include those where the ribose ring structure is modified. These modifications include replacement with a hexose ring (HNA), a bicyclic ring having a biradicle bridge between the C2 and C4 carbons on the ribose ring (e.g., locked nucleic acids (LNA)), or an unlinked ribose ring which typically lacks a bond between the C2 and C3 carbons (e.g., UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids. In some embodiments, the modified sugar is a bicyclic sugar. Further nucleoside modifications are described in U.S. Pat. No. 11,241,451, which can be further employed as described herein.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl, 2′-O-methyl (2′-OMe), 2′-alkoxy, 2′-O-methoxyethyl-oligos (MOE), 2′-amino, 2′-Fluoro, and 2′-F-ANA nucleoside. The 2′ substituted modified nucleosides can be DNA or RNA. In some embodiments, the modified sugar comprises a 2′-O-methoxyethyl (MOE) group. In some embodiments, the modified sugar comprises a 2′-O-alkyl group. In some embodiments, the modified sugar comprises a 2′-O-methyl (2′-OMe) group. In some embodiments, the modified sugar comprises a 2′-alkoxy group. In some embodiments, the modified sugar comprises a 2′-O-methoxyethyl-oligos (MOE) group. In some embodiments, the modified sugar comprises a 2′-amino group. In some embodiments, the modified sugar comprises a 2′-Fluoro group. In some embodiments, the modified sugar comprises a 2′-F-ANA group.

In certain additional embodiments, 2′-substituent groups are selected from among: halo, allyl, amino, azido, SH, CN, OCN, CF3, OCF3, O—C1-Cm alkoxy, O—C1-C10 substituted alkoxy, O—C1-Cm alkyl, O—C1-C10 substituted alkyl, 5-alkyl, N(Rm) alkyl, O-alkenyl, S-alkenyl, N(Rm)-alkenyl, O-alkynyl, 5-alkynyl, N(Rm) alkynyl, O-alkylenyl-O-alkyl, alkynyl, alkaryl, aralkyl, O-alkaryl, O-aralkyl, O(CH2)25CH3, O(CH2)2ON(Rm)(R.) or OCH2C(═O)—N(Rm)(R.), where each Rm and R. is, independently, H, an amino protecting group, or substituted or unsubstituted C1-C10 alkyl groups. Certain embodiments of such 2′-substituent groups can be further substituted with one or more substituent groups independently selected from among: hydroxyl, amino, alkoxy, carboxy, benzyl, phenyl, nitro (NO2), thiol, thioalkoxy, thioalkyl, halogen, alkyl, aryl, alkenyl and alkynyl

In some embodiments, the modified antisense oligonucleotide comprises one or more modified sugars. In some embodiments, the modified antisense oligonucleotide comprises only modified sugars. In certain embodiments, the modified antisense oligo comprises greater than 10%, 25%, 50%, 75%, or 90% modified nucleosides.

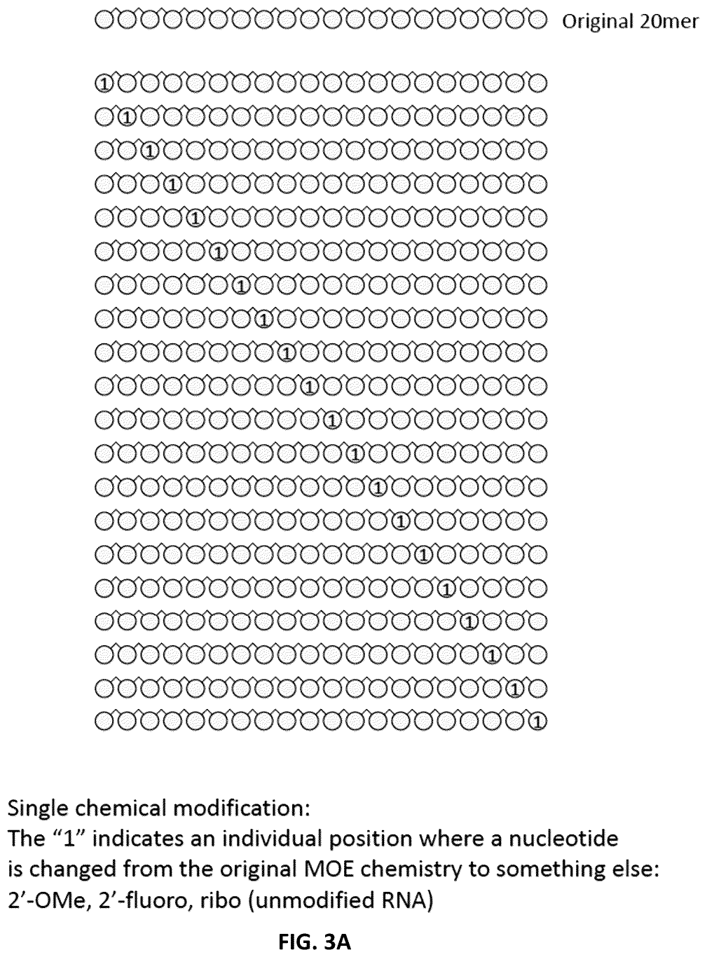

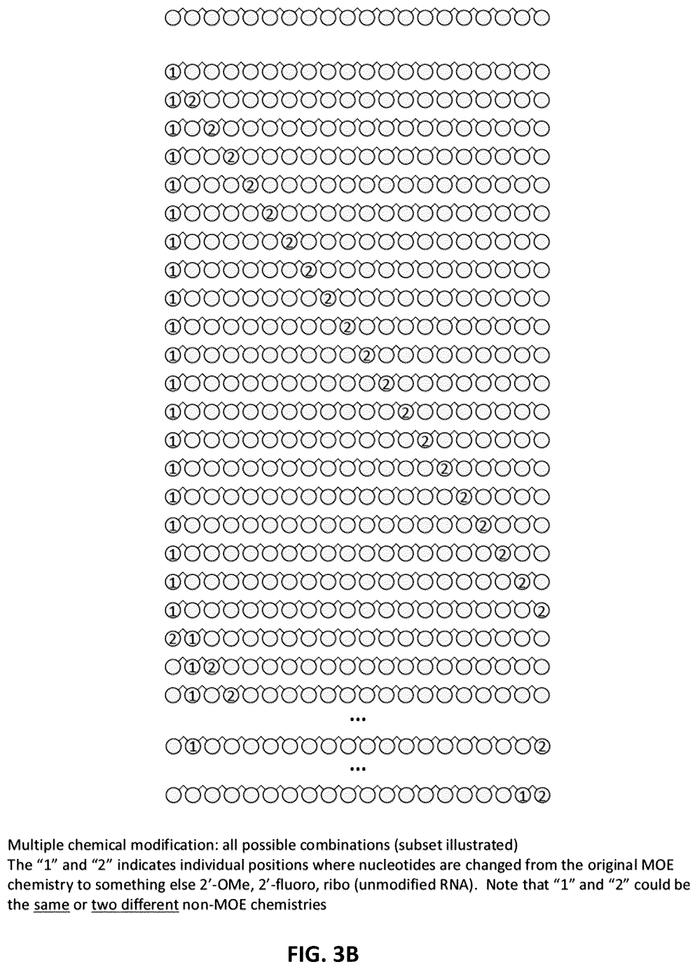

FIG. 3 demonstrates exemplary nucleotide positions (circles) that can comprise the modified nucleoside(s). In certain embodiments, the modified antisense oligonucleotides (ASOs) comprise 10% modified nucleosides to 100% modified nucleosides. In certain embodiments, the modified antisense oligonucleotides (ASOs) comprise 10% modified nucleosides to 20% modified nucleosides, 10% modified nucleosides to 30% modified nucleosides, 10% modified nucleosides to 40% modified nucleosides, 10% modified nucleosides to 50% modified nucleosides, 10% modified nucleosides to 60% modified nucleosides, 10% modified nucleosides to 70% modified nucleosides, 10% modified nucleosides to 80% modified nucleosides, 10% modified nucleosides to 90% modified nucleosides, 10% modified nucleosides to 100% modified nucleosides, 20% modified nucleosides to 30% modified nucleosides, 20% modified nucleosides to 40% modified nucleosides, 20% modified nucleosides to 50% modified nucleosides, 20% modified nucleosides to 60% modified nucleosides, 20% modified nucleosides to 70% modified nucleosides, 20% modified nucleosides to 80% modified nucleosides, 20% modified nucleosides to 90% modified nucleosides, 20% modified nucleosides to 100% modified nucleosides, 30% modified nucleosides to 40% modified nucleosides, 30% modified nucleosides to 50% modified nucleosides, 30% modified nucleosides to 60% modified nucleosides, 30% modified nucleosides to 70% modified nucleosides, 30% modified nucleosides to 80% modified nucleosides, 30% modified nucleosides to 90% modified nucleosides, 30% modified nucleosides to 100% modified nucleosides, 40% modified nucleosides to 50% modified nucleosides, 40% modified nucleosides to 60% modified nucleosides, 40% modified nucleosides to 70% modified nucleosides, 40% modified nucleosides to 80% modified nucleosides, 40% modified nucleosides to 90% modified nucleosides, 40% modified nucleosides to 100% modified nucleosides, 50% modified nucleosides to 60% modified nucleosides, 50% modified nucleosides to 70% modified nucleosides, 50% modified nucleosides to 80% modified nucleosides, 50% modified nucleosides to 90% modified nucleosides, 50% modified nucleosides to 100% modified nucleosides, 60% modified nucleosides to 70% modified nucleosides, 60% modified nucleosides to 80% modified nucleosides, 60% modified nucleosides to 90% modified nucleosides, 60% modified nucleosides to 100% modified nucleosides, 70% modified nucleosides to 80% modified nucleosides, 70% modified nucleosides to 90% modified nucleosides, 70% modified nucleosides to 100% modified nucleosides, 80% modified nucleosides to 90% modified nucleosides, 80% modified nucleosides to 100% modified nucleosides, or 90% modified nucleosides to 100% modified nucleosides. In certain embodiments, the modified antisense oligonucleotides (ASOs) comprise 10% modified nucleosides, 20% modified nucleosides, 30% modified nucleosides, 40% modified nucleosides, 50% modified nucleosides, 60% modified nucleosides, 70% modified nucleosides, 80% modified nucleosides, 90% modified nucleosides, or 100% modified nucleosides. In certain embodiments, the modified antisense oligonucleotides (ASOs) comprise at least 10% modified nucleosides, 20% modified nucleosides, 30% modified nucleosides, 40% modified nucleosides, 50% modified nucleosides, 60% modified nucleosides, 70% modified nucleosides, 80% modified nucleosides, or 90% modified nucleosides. In certain embodiments, the modified antisense oligonucleotides (ASOs) comprise at most 20% modified nucleosides, 30% modified nucleosides, 40% modified nucleosides, 50% modified nucleosides, 60% modified nucleosides, 70% modified nucleosides, 80% modified nucleosides, 90% modified nucleosides, or 100% modified nucleosides.

In certain embodiments, the modified antisense oligonucleotides (ASOs) comprise 1 modified nucleoside to 23 modified nucleosides. In certain embodiments, the modified antisense oligonucleotides (ASOs) comprise 1 modified nucleoside to 2 modified nucleosides, 1 modified nucleoside to 3 modified nucleosides, 1 modified nucleoside to 4 modified nucleosides, 1 modified nucleoside to 5 modified nucleosides, 1 modified nucleoside to 10 modified nucleosides, 1 modified nucleoside to 12 modified nucleosides, 1 modified nucleoside to 15 modified nucleosides, 1 modified nucleoside to 18 modified nucleosides, 1 modified nucleoside to 20 modified nucleosides, 1 modified nucleoside to 22 modified nucleosides, 1 modified nucleoside to 23 modified nucleosides, 2 modified nucleosides to 3 modified nucleosides, 2 modified nucleosides to 4 modified nucleosides, 2 modified nucleosides to 5 modified nucleosides, 2 modified nucleosides to 10 modified nucleosides, 2 modified nucleosides to 12 modified nucleosides, 2 modified nucleosides to 15 modified nucleosides, 2 modified nucleosides to 18 modified nucleosides, 2 modified nucleosides to 20 modified nucleosides, 2 modified nucleosides to 22 modified nucleosides, 2 modified nucleosides to 23 modified nucleosides, 3 modified nucleosides to 4 modified nucleosides, 3 modified nucleosides to 5 modified nucleosides, 3 modified nucleosides to 10 modified nucleosides, 3 modified nucleosides to 12 modified nucleosides, 3 modified nucleosides to 15 modified nucleosides, 3 modified nucleosides to 18 modified nucleosides, 3 modified nucleosides to 20 modified nucleosides, 3 modified nucleosides to 22 modified nucleosides, 3 modified nucleosides to 23 modified nucleosides, 4 modified nucleosides to 5 modified nucleosides, 4 modified nucleosides to 10 modified nucleosides, 4 modified nucleosides to 12 modified nucleosides, 4 modified nucleosides to 15 modified nucleosides, 4 modified nucleosides to 18 modified nucleosides, 4 modified nucleosides to 20 modified nucleosides, 4 modified nucleosides to 22 modified nucleosides, 4 modified nucleosides to 23 modified nucleosides, 5 modified nucleosides to 10 modified nucleosides, 5 modified nucleosides to 12 modified nucleosides, 5 modified nucleosides to 15 modified nucleosides, 5 modified nucleosides to 18 modified nucleosides, 5 modified nucleosides to 20 modified nucleosides, 5 modified nucleosides to 22 modified nucleosides, 5 modified nucleosides to 23 modified nucleosides, 10 modified nucleosides to 12 modified nucleosides, 10 modified nucleosides to 15 modified nucleosides, 10 modified nucleosides to 18 modified nucleosides, 10 modified nucleosides to 20 modified nucleosides, 10 modified nucleosides to 22 modified nucleosides, 10 modified nucleosides to 23 modified nucleosides, 12 modified nucleosides to 15 modified nucleosides, 12 modified nucleosides to 18 modified nucleosides, 12 modified nucleosides to 20 modified nucleosides, 12 modified nucleosides to 22 modified nucleosides, 12 modified nucleosides to 23 modified nucleosides, 15 modified nucleosides to 18 modified nucleosides, 15 modified nucleosides to 20 modified nucleosides, 15 modified nucleosides to 22 modified nucleosides, 15 modified nucleosides to 23 modified nucleosides, 18 modified nucleosides to 20 modified nucleosides, 18 modified nucleosides to 22 modified nucleosides, 18 modified nucleosides to 23 modified nucleosides, 20 modified nucleosides to 22 modified nucleosides, 20 modified nucleosides to 23 modified nucleosides, or 22 modified nucleosides to 23 modified nucleosides. In certain embodiments, the modified antisense oligonucleotides (ASOs) comprise 1 modified nucleoside, 2 modified nucleosides, 3 modified nucleosides, 4 modified nucleosides, 5 modified nucleosides, 10 modified nucleosides, 12 modified nucleosides, 15 modified nucleosides, 18 modified nucleosides, 20 modified nucleosides, 22 modified nucleosides, or 23 modified nucleosides. In certain embodiments, the modified antisense oligonucleotides (ASOs) comprise at least 1 modified nucleoside, 2 modified nucleosides, 3 modified nucleosides, 4 modified nucleosides, 5 modified nucleosides, 10 modified nucleosides, 12 modified nucleosides, 15 modified nucleosides, 18 modified nucleosides, 20 modified nucleosides, or 22 modified nucleosides. In certain embodiments, the modified antisense oligonucleotides (ASOs) comprise at most 2 modified nucleosides, 3 modified nucleosides, 4 modified nucleosides, 5 modified nucleosides, 10 modified nucleosides, 12 modified nucleosides, 15 modified nucleosides, 18 modified nucleosides, 20 modified nucleosides, 22 modified nucleosides, or 23 modified nucleosides.

In some embodiments, the modified antisense oligonucleotide comprises both inter-nucleoside linker modifications and nucleoside modifications.

### Pharmaceutical Compositions

Further provided herein are pharmaceutical compositions comprising any of the disclosed antisense oligonucleotides and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments, the oligonucleotide is used in the pharmaceutically acceptable diluent at a concentration of 50-300 UM solution. In some embodiments, the oligonucleotide, as described, is administered at a dose of 10-1000 ug.

The modified antisense oligonucleotides or oligonucleotide conjugates of the disclosure may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, the extent of disease, or dose to be administered.

### Methods of Use

The modified antisense oligonucleotides (ASOs) provided herein are useful for targeting a FOXG1 nucleic acid encoding a functional FOXG1 protein, wherein an antisense oligonucleotide inhibits translation inhibition, interferes with upstream open reading frames (uORFs), inhibits RNA degradation, and/or increases RNA stability to ultimately increase protein expression of a functional FOXG1 protein. According, the modified antisense oligonucleotides targeting are further useful in methods for increasing the expression and/or amount of functional FOXG1 in a cell (e.g., an amount of functional FOXG1 mRNA or protein). Accordingly, provided herein are methods of modulating expression of a FOXG1 in a cell, comprising contacting the cell with a composition comprising an antisense oligonucleotide complementary to a target nucleic acid sequence of a FOXG1 nucleic acid.

Further provided, are methods of treating or ameliorating a FOXG1 disease or disorder in an individual having, or at risk of having, the FOXG1 disease or disorder, comprising administering to the individual an antisense oligonucleotide, wherein the modified antisense oligonucleotide comprises a sequence complementary to a target sequence of the FOXG1 nucleic acid, thereby treating or ameliorating a FOXG1 disease in the individual.

Generally, cells of interest include neuronal cells and/or cells associated with the brain or brain development. In some embodiments, the cell is located in a brain of an individual. In some embodiments, the cell is a neural cell. In some embodiments, the individual is a human. In certain embodiments, the human is an unborn human.

The modified antisense oligonucleotides (ASOs) and methods are particularly useful for increasing the expression and/or amount of functional FOXG1 (e.g., an amount of functional FOXG1 mRNA or protein) in a cell and/or individual comprising a mutated or deleted FOXG1 allele. In some embodiments, the cell and/or individual comprises a mutated FOXG1 gene. In some embodiments the individual has been diagnosed with or at risk of a FOXG1 disease or disorder. In some embodiments, the FOXG1 disease o disorder is FOXG1 syndrome.

In some embodiments, modulating expression comprises increasing expression of a FOXG1 protein in the cell. In some embodiments, modulating expression comprises increasing stability or half-life of the FOXG1 nucleic acid in the cell. In some embodiments, modulating expression comprises increasing translation of a FOXG1 protein in the cell.

In order to achieve effective targeting of a FOXG1 RNA (e.g., messenger RNA), the modified antisense oligonucleotides disclosed herein (ASOs) comprise a sequence complementary to a sequence of the FOXG1 RNA, wherein the complementary sequence binds and/or hybridizes to a sequence of the FOXG1 RNA. For example, mRNA transcripts comprise a 5′ untranslated region (5′ UTR) and a 3′ untranslated region (3′ UTR). The modified antisense oligonucleotides (ASOs) disclosed herein target the 5′ UTR or the 3′ UTR of a FOXG1 mRNA transcript. In order to achieve targeting of the 5′ UTR or 3′ UTR, the modified antisense oligonucleotide (ASOs) comprise a sequence complementary to a target sequence is located at the 5′ UTR or the 3′ UTR of the FOXG1 mRNA. In some embodiments, the target sequence is located at or within the 5′ UTR. In some embodiments, the target sequence is located at or within the 3′ UTR. In certain embodiments, the modified antisense oligonucleotide targeting the 3′ UTR comprises a nucleobase sequence complementary to a sequence within NM_005249.5_2000-2200_as region or NM_005249.5_2900-3000_as of the FOXG1 nucleic acid. In certain embodiments, the modified antisense oligonucleotide targeting the 3′ UTR comprises a nucleobase sequence complementary to a sequence within NM_005249.5_2000-2100_as region of the FOXG1 nucleic acid. In some embodiments, the modified antisense oligonucleotides are included in an ASO composition comprising more than one ASO. In certain the embodiments, the ASO composition comprises 2, 3, 4, 5 or more ASOs.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: Tablets, Marcel Dekker, NY, 1990; Lieberman, et al. (eds.) Pharmaceutical Dosage Forms: Disperse Systems, Inc., New York, N.Y., 2000).

Compositions comprising antisense oligonucleotides (ASOs), as disclosed herein, can be provided by doses at intervals of, e.g., one day, one week, or 1-7 times per week. A specific dose protocol is one involving the maximal dose or dose frequency that avoids significant undesirable side effects.

The disclosed antisense oligonucleotides or pharmaceutical compositions thereof can be administered topically (such as, to the skin, inhalation, ophthalmic or otic) or enterally (such as, orally or through the gastrointestinal tract) or parenterally (such as, intravenous, subcutaneous, intra-muscular, intracerebral, intracerebroventricular or intrathecal). In some embodiments, the modified antisense oligonucleotide or pharmaceutical compositions thereof are administered by a parenteral route including intravenous, intraarterial, subcutaneous, intraperitoneal or intramuscular injection or infusion, intrathecal or intracranial, e.g., intracerebral or intraventricular, administration. In some embodiments, the active oligonucleotide or oligonucleotide conjugate is administered intravenously.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “FOXG1,” as used herein, generally refers to the gene and gene products that encode a member of the fork-head transcription factor family. The encoded protein, which functions as a transcriptional repressor, is highly expressed in neural tissues during brain development. Mutations at this locus have been associated with Rett syndrome and a diverse spectrum of neurodevelopmental disorders defined as part of FOXG1 syndrome. Depending on the context of its use, “FOXG1” can refer to the FOXG1 gene, a FOXG1 deoxyribonucleic acid molecule (DNA), a FOXG1 ribonucleic acid molecule (RNA), or a FOXG1 protein. The mRNA sequence of FOXG1 is described in “NM_005249.5→NP_005240.3 forkhead box protein G1” or “accession number NM_005249.5” or the mRNA encoded by “NCBI GENE ID: 2290”. A functional FOXG1 protein describes the wild-type or unmutated FOXG1 gene, mRNA, and/or protein. Generally, “FOXG1” refers to a functional ‘FOXG1” gene or gene product, having normal function/activity within a cell. Deletions or mutations or variants of FOXG1 are indicative of non-functional FOXG1 variants having reduced, inhibited, or ablated FOXG1 function. As disclosed herein, the compositions and methods disclosed herein are primarily concerned with modulating or increasing or restoring an amount of FOXG1 (i.e., functional FOXG1) in a cell and/or individual.

The term “oligonucleotide,” as used herein, generally refers to a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the disclosure is man-made, and is chemically synthesized, and is typically purified or isolated. The oligonucleotide disclosed may comprise one or more modified nucleosides or nucleotides.

The term “antisense oligonucleotide,” as used herein, refers to oligonucleotides capable of modulating expression of a target gene by hybridizing to a target nucleic acid, in particular to a contiguous sequence on a target nucleic acid. Preferably, the modified antisense oligonucleotides of the present disclosure are single stranded. In some embodiments, the modified antisense oligonucleotide is single stranded.

The term “modified oligonucleotide” refers to an oligonucleotide comprising one or more sugar-modified nucleosides, modified nucleobases, and/or modified inter-nucleoside linkers.

The term “modified nucleoside” or “nucleoside modification,” as used herein, refers to nucleosides modified as compared to the equivalent DNA or RNA nucleoside by the introduction of one or more modifications of the sugar moiety or the (nucleo) base moiety. In some embodiments, the modified nucleoside comprise a modified sugar moiety. The term modified nucleoside may also be used herein interchangeably with the term “nucleoside analogue” or modified “units” or modified “monomers”.

The term “modified inter-nucleoside linkage” is refers to linkers other than phosphodiester (PO) linkers, that covalently couples two nucleosides together. Nucleotides with modified inter-nucleoside linkage are also termed “modified nucleotides”. In some embodiments, the modified inter-nucleoside linkage increases the nuclease resistance of the oligonucleotide compared to a phosphodiester linkage. For naturally occurring oligonucleotides, the inter-nucleoside linkage includes phosphate groups creating a phosphodiester bond between adjacent nucleosides. Modified inter-nucleoside linkers are particularly useful in stabilizing oligonucleotides for in vivo use and may serve to protect against nuclease cleavage at regions of DNA or RNA nucleosides.

The term “nucleobase” includes the purine (e.g., adenine and guanine) and pyrimidine (e.g., uracil, thymine and cytosine) moiety present in nucleosides and nucleotides which form hydrogen bonds in nucleic acid hybridization. The term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants.

A nucleobase moiety can be modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g., A, T, G, C or U, wherein each letter may optionally include modified nucleobases of equivalent function. For example, in the exemplified oligonucleotides, the nucleobase moieties are selected from A, T, G, C, and 5-methyl cytosine. In some embodiments, the cytosine nucleobases in a 5′cg3′ motif is 5-methyl cytosine.

The term “hybridizing” or “hybridizes” or “targets” or “binds” describes two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid.

The oligonucleotide comprises a contiguous nucleotide region which is complementary to or hybridizes to a sub-sequence or region of the target nucleic acid molecule. The term “target sequence” as used herein refers to a sequence of nucleotides present in the target nucleic acid which comprises the nucleobase sequence which is complementary to the contiguous nucleotide region or sequence of the oligonucleotide of the disclosure. In some embodiments, the target sequence consists of a region on the target nucleic acid which is complementary to the contiguous nucleotide region or sequence of the oligonucleotide of the present disclosure. In some embodiments the target sequence is longer than the complementary sequence of a single oligonucleotide, and may, for example represent a preferred region of the target nucleic acid which may be targeted by several oligonucleotides of the present disclosure.

The oligonucleotide of the present disclosure comprises a contiguous nucleotide region which is complementary to a FOXG1 target nucleic acid, such as a target sequence of FOXG1.

The oligonucleotide comprises a contiguous nucleotide region of at least 10 nucleotides which is complementary to or hybridizes to a target sequence present in the target nucleic acid molecule. The contiguous nucleotide region (and therefore the target sequence) comprises of at least 10 contiguous nucleotides, such as 11, 12, 13, 14, 15, 16, 17, 18, 19, 20, 21, 22, 23, 24, 25, 30 contiguous nucleotides, such as from 15-30, such as from 18-23 contiguous nucleotides.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

As used in the specification and claims, the singular forms “a”, “an” and “the” include plural references unless the context clearly dictates otherwise. For example, the term “a sample” includes a plurality of samples, including mixtures thereof.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the present disclosure.

### Example 1: Cellular Modulation of FOXG1 Expression by ASOs

The designed antisense oligonucleotides (ASOs) targeting 3′ UTR region of a FOXG1 mRNA were tested for the ability to modulate (e.g., increase) FOXG1 expression in cells.

### Cells:

Cells were cultured in EMEM supplemented to contain 10% fetal calf serum, and 100U/ml Penicillin/100 ug/ml Streptomycin at 37° C. in an atmosphere with 5% CO₂ in a humidified incubator. For transfection of cells with ASOs, cells were generally seeded at a density of 15,000 cells/well into 96-well tissue culture plates (#655180, GBO, Germany).

### Transfection of ASOs:

Transfection of ASOs was carried out with lipofectamine for reverse transfection with 0.5 μL lipofectamine per well.

The single dose screen was performed with ASOs in quadruplicates at 50 nM, with two ASOs targeting AHSA1 (one 2′-O-methoxyethyl (MOE) and one 2′-O-methyl (oMe) ASO) and a siRNA targeting RLuc as unspecific controls and a mock transfection. After 24h of incubation with ASOs, medium was removed and cells were lysed in 150 μl Medium-Lysis Mixture (1 volume lysis mixture, 2 volumes cell culture medium) and then incubated at 53° C. for 30 minutes.

The two Ahsa1-ASOs (one 2′-oMe-modified and one 2′-O-methoxyethyl (MOE MOE)-modified) served at the same time as unspecific controls for respective target mRNA expression and as a positive control to analyze transfection efficiency with regards to Ahsa1 mRNA level. By hybridization with an Ahsa1 probe set, the mock transfected wells served as controls for Ahsa1 mRNA level. Transfection efficiency for each 96-well plate and both doses in the dual dose screen were calculated by relating Ahsa1-level with Ahsa1-ASO (normalized to GapDH) to Ahsa1-level obtained with mock controls.

Detection of FOXG1 mRNA:

QuantiGene detection was used to determine FOXG1 mRNA expression in cells lysates. In short, the QuantiGene assay directly measures target RNAs captured through probe hybridization and quantified through branched DNA technology that amplifies the signal. The signal is read using a Luminex or a luminometer for single targets. The assay measures RNA at the sample source, the assay avoids biases and variability inherent to extraction techniques and enzymatic manipulations. In addition, this direct measurement helps overcome issues with transcript degradation typically found in samples such as FFPE.

For the detection of FOXG1 mRNA, a Quantigene-Singleplex assay (1.0 for GapDH and 2.0 for FoxG1) was performed according to manufacturer's instructions (ThermoFisher, Germany). Luminescence was read using 1420 Luminescence Counter (WALLAC VICTOR Light, Perkin Elmer, Rodgau-Jügesheim, Germany) following 30 minutes incubation at RT in the dark. The probe sets used for FOXG1 mRNA detection are set forth in Table 1 (Human FoxG1 QG2.0 probe set (Accession #NM_005249): Oligosequences “CEs” and “LEs” are depicted without the proprietary parts of their sequences. Cross reactivity with the cyno sequence was obtained by adding additional probes). Control GapDH probe sets are set forth in Table 4 (Human GapDH QG1.0 probe set (Accession #NM_002046): Oligosequences “CEs” and “LEs” are depicted without the proprietary parts of their sequences.).

TABLE 1
Human FoxG1 QG2.0 probe set (Accession #NM_005249)

| Oligo name | sequence 5′-3′ | accession#, position & |
|---|---|---|
| QG2_hsFoxG1_1 | ggccagcttggcccg | NM_005249.1334.1348.LE |
| QG2_hsFoxG1_2 | gcgcaccgcgcttgaa | NM_005249.1349.1364.LE |
| QG2_hsFoxG1_3 | gccggtggaggtgaggc | NM_005249.1365.1381.CE |
| QG2_hsFoxG1_4 | cgcggtccatgaaggtgag | NM_005249.1382.1400.LE |
| QG2_hsFoxG1_5 | gccagtagagggagccgg | NM_005249.1401.1418.LE |
| QG2_hsFoxG1_6 | gacaggaagggcgacatgg | NM_005249.1419.1437.BL |
| QG2_hsFoxG1_7 | gcgggggtggtgcagg | NM_005249.1438.1453.BL |
| QG2_hsFoxG1_8 | tgtaactcaaagtgctgctggc | NM_005249.1454.1475.CE |
| QG2_hsFoxG1_9 | gccgacgtggtgccgt | NM_005249.1476.1491.LE |
| QG2_hsFoxG1_10 | atggggtggctggggtag | NM_005249.1492.1509.LE |
| QG2_hsFoxG1_11 | tcaacacggagctgtagggc | NM_005249.1510.1529.CE |
| QG2_hsFoxG1_12 | gttgcccagcgagttctgag | NM_005249.1530.1549.LE |
| QG2_hsFoxG1_13 | gcggtggagaaggagtggtt | NM_005249.1550.1569.LE |
| QG2_hsFoxG1_14 | ccacgctcaggccgttg | NM_005249.1570.1586.BL |
| QG2_hsFoxG1_15 | cccgttgaccagccggt | NM_005249.1587.1603.CE |
| QG2_hsFoxG1_16 | cgtggcgtacgggatctc | NM_005249.1604.1621.LE |
| QG2_hsFoxG1_17 | gcggccgtgaggtggtg | NM_005249.1622.1638.LE |
| QG2_hsFoxG1_18 | gaggcggctagcgcg | NM_005249.1639.1653.CE |
| QG2_hsFoxG1_19 | caggccgcagggcacc | NM_005249.1654.1669.LE |
| QG2_hsFoxG1_20 | ccagagcagggcaccga | NM_005249.1670.1686.LE |
| QG2_hsFoxG1_21 | caggggttgagggagtaggtc | NM_005249.1687.1707.CE |
| QG2_hsFoxG1_22 | gcgagcaggttgacggag | NM_005249.1708.1725.LE |
| QG2_hsFoxG1_23 | gaaaaagtaactggtctggccc | NM_005249.1726.1747.LE |
| QG2_hsFoxG1_24 | ggtgcgggacgtgggg | NM_005249.1748.1763.CE |
| QG2_hsFoxG1_25 | tgctctgcgaagtcattgacg | NM_005249.1764.1784.LE |
| QG2_hsFoxG1_26 | ggcgctcatggacgtgc | NM_005249.1785.1801.LE |
| QG2_hsFoxG1_27 | aggaggacgcggccct | NM_005249.1802.1817.CE |

TABLE 2
Human GapDH QG1.0 probe set (Accession #NM_002046)

| Oligo name | sequence 5′-3′ | accession#, position & |
|---|---|---|
| QG1_hsGAP_1 | gaatttgccatgggtggaat | NM_002046.252.271.CE |
| QG1_hsGAP_2 | ggagggatctcgctcctgga | NM_002046.333.352.CE |
| QG1_hsGAP_3 | ccccagccttctccatggt | NM_002046.413.431.CE |
| QG1_hsGAP_4 | gctcccccctgcaaatgag | NM_002046.432.450.CE |
| QG1_hsGAP_5 | agccttgacggtgccatg | NM_002046.272.289.LE |
| QG1_hsGAP_6 | gatgacaagcttcccgttctc | NM_002046.290.310.LE |
| QG1_hsGAP_7 | agatggtgatgggatttccatt | NM_002046.311.332.LE |
| QG1_hsGAP_8 | gcatcgccccacttgatttt | NM_002046.353.372.LE |
| QG1_hsGAP_9 | cacgacgtactcagcgcca | NM_002046.373.391.LE |
| QG1_hsGAP_10 | ggcagagatgatgacccttttg | NM_002046.451.472.LE |
| QG1_hsGAP_11 | ggtgaagacgccagtggactc | NM_002046.392.412.BL |

### Example 2: Cellular Increase of FOXG1 Expression by Select ASOs in Cells

The designed antisense oligonucleotides (ASOs) targeting a FOXG1 mRNA were further tested for the ability to modulate (e.g., increase) FOXG1 expression in cells.

### Transfection of ASOs and FOXG1 Quantification:

In cells, transfection was performed with ASOs at concentrations of 50 nM and 10 nM in replicate. After 24h of incubation with ASOs, medium was removed, the cells were lysed, and QuantiGene detection was used to determine FOXG1 mRNA expression in cells lysates.

### Modulation of FOXG1 Expression by ASOs:

Table 3 shows 2′-O-methoxyethyl (MOE) chemistry ASO targets associated with an increase in FOXG1 expression in HEK293, relative to mean of mock transfection control. ASOs are arranged by and listed in order of start position in FOXG1 mRNA (RefSeq NM_005249.5).

TABLE 3
ASO targets resulting in up-regulation of FOXG1 mRNA in cells
Oligo (Position in FOXG1 mRNA)

NM_005249.5_2061-2080
NM_005249.5_2062-2081
NM_005249.5_2063-2082
NM_005249.5_2064-2083
NM_005249.5_2065-2084
NM_005249.5_2961-2980
NM_005249.5_2962-2981
NM_005249.5_2963-2982
NM_005249.5_2964-2983
NM_005249.5_2965-2984
NM_005249.5_2966-2985
NM_005249.5_2967-2986
NM_005249.5_2968-2987
NM_005249.5_2969-2988
NM_005249.5_2970-2989
NM_005249.5_2971-2990
NM_005249.5_2973-2992
NM_005249.5_2976-2995
NM_005249.5_2977-2996
NM_005249.5_2978-2997
NM_005249.5_2983-3002
NM_005249.5_2984-3003
NM_005249.5_2985-3004
NM_005249.5_2986-3005
NM_005249.5_2987-3006
NM_005249.5_2990-3009
NM_005249.5_2991-3010
NM_005249.5_2992-3011
NM_005249.5_2993-3012
NM_005249.5_2994-3013
NM_005249.5_2995-3014
NM_005249.5_2996-3015
NM_005249.5_2997-3016
NM_005249.5_2998-3017
NM_005249.5_2999-3018
NM_005249.5_3000-3019
NM_005249.5_2061-2080
NM_005249.5_2062-2081
NM_005249.5_2063-2082
NM_005249.5_2064-2083
NM_005249.5_2065-2084
NM_005249.5_2961-2980
NM_005249.5_2962-2981
NM_005249.5_2963-2982
NM_005249.5_2964-2983
NM_005249.5_2965-2984
NM_005249.5_2966-2985
NM_005249.5_2967-2986
NM_005249.5_2968-2987
NM_005249.5_2969-2988
NM_005249.5_2970-2989
NM_005249.5_2971-2990
NM_005249.5_2973-2992
NM_005249.5_2976-2995
NM_005249.5_2977-2996
NM_005249.5_2978-2997
NM_005249.5_2983-3002
NM_005249.5_2984-3003
NM_005249.5_2985-3004
NM_005249.5_2986-3005
NM_005249.5_2987-3006
NM_005249.5_2990-3009
NM_005249.5_2991-3010
NM_005249.5_2992-3011
NM_005249.5_2993-3012
NM_005249.5_2994-3013
NM_005249.5_2995-3014
NM_005249.5_2996-3015
NM_005249.5_2997-3016
NM_005249.5_2998-3017
NM_005249.5_2999-3018
NM_005249.5_3000-3019

Table 4 shows ASO coverage of the FOXG1 mRNA and data for select ASOs associated with the modulation of FOXG1 expression in CFF-STTG1 and SW1783 cell lines. ASOs are arranged by and listed in order of start position in FOXG1 mRNA (RefSeq NM_005249.5).

TABLE 4
ASO targets resulting in upregulation of
FOXG1 mRNA in CFF-STTG1 and SW1783 cells
Oligo (Position in FoxG1 mRNA)

NM_005249.5_2061-2080_as
NM_005249.5_2064-2083_as
NM_005249.5_2965-2984_as
NM_005249.5_2967-2986_as
NM_005249.5_2968-2987_as
NM_005249.5_2995-3014_as
NM_005249.5_2996-3015_as
NM_005249.5_2061-2080_as
NM_005249.5_2064-2083_as
NM_005249.5_2965-2984_as
NM_005249.5_2967-2986_as
NM_005249.5_2968-2987_as
NM_005249.5_2995-3014_as
NM_005249.5_2996-3015_as
NM_005249.5_2061-2080_as
NM_005249.5_2064-2083_as
NM_005249.5_2965-2984_as
NM_005249.5_2967-2986_as
NM_005249.5_2968-2987_as
NM_005249.5_2995-3014_as
NM_005249.5_2996-3015_as
NM_005249.5_2061-2080_as
NM_005249.5_2064-2083_as
NM_005249.5_2965-2984_as
NM_005249.5_2967-2986_as
NM_005249.5_2968-2987_as
NM_005249.5_2995-3014_as
NM_005249.5_2996-3015_as

### Example 3: Cellular Increase of FOXG1 Expression by Select Modified ASOs in Cells

Three exemplary designed antisense oligonucleotide (ASO) sequences targeting a FOXG1 mRNA were modified, generating 189 (63×3) modified ASOs. These 189 modified ASOs were tested for the ability to modulate (e.g., increase) FOXG1 expression in cells.

### Modified ASOs:

Each of the following three ASOs was selected as the base sequence for modification:

TABLE 5
Base ASO Sequences

| Name | Base Sequence | Base SEQ NO. |
|---|---|---|
| NM_005249.5_2061-2080_as_MOE | AACGTACAGAAATGGGAGGG | 100 |
| NM_005249.5_2062-2081_as_MOE | AAACGTACAGAAATGGGAGG | 101 |
| NM_005249.5_2965-2984_as_MOE | TAAATTTTAGTTTGGCTGAA | 284 |

Base modifications made to the three base sequences were as follows:

TABLE 6
Base modification code

| Code | Modified Base |
|---|---|
| Am | 2′-O-methoxy-ethyl A |
| Tm | 2′-O-methoxy-ethyl T |
| Cm | 2′-O-methoxy-ethyl C |
| Gm | 2′-O-methoxy-ethyl G |
| Ab | LNA A |
| Tb | LNA T |
| Cb | LNA C |
| Gb | LNA G |
| s | phosphorothioate linkage |

### Generated Modified ASOs:

TABLE 6
NM_005249.5_2061-2080 as MOE Modification Sequences

| Name | Sequence |
|---|---|
| NM_005249.5_2965- | TmsAmsAmsAmsTmsTmsTmsTmsAmsGmsTmsTmsTmsGmsGmsCmsTm |
| 2984_as_MOE | sGmsAmsAm |
| NM_005249.5_2965- | TbsAmsAmsAmsTmsTmsTmsTmsAmsGmsTmsTmsTmsGmsGmsCmsTm |
| 2984_as_MOE_L1 | sGmsAmsAm |
| NM_005249.5_2965- | TmsAbsAmsAmsTmsTmsTmsTmsAmsGmsTmsTmsTmsGmsGmsCmsTm |
| 2984_as_MOE_L2 | sGmsAmsAm |
| NM_005249.5_2965- | TmsAmsAbsAmsTmsTmsTmsTmsAmsGmsTmsTmsTmsGmsGmsCmsTm |
| 2984_as_MOE_L3 | sGmsAmsAm |
| NM_005249.5_2965- | TmsAmsAmsAbsTmsTmsTmsTmsAmsGmsTmsTmsTmsGmsGmsCmsTm |
| 2984_as_MOE_L4 | sGmsAmsAm |
| NM_005249.5_2965- | TmsAmsAmsAmsTbsTmsTmsTmsAmsGmsTmsTmsTmsGmsGmsCmsTm |
| 2984_as_MOE_L5 | sGmsAmsAm |
| NM_005249.5_2965- | TmsAmsAmsAmsTmsTbsTmsTmsAmsGmsTmsTmsTmsGmsGmsCmsTm |
| 2984_as_MOE_L6 | sGmsAmsAm |
| NM_005249.5_2965- | TmsAmsAmsAmsTmsTmsTbsTmsAmsGmsTmsTmsTmsGmsGmsCmsTm |
| 2984_as_MOE_L7 | sGmsAmsAm |
| NM_005249.5_2965- | TmsAmsAmsAmsTmsTmsTmsTbsAmsGmsTmsTmsTmsGmsGmsCmsTm |
| 2984_as_MOE_L8 | sGmsAmsAm |
| NM_005249.5_2965- | TmsAmsAmsAmsTmsTmsTmsTmsAbsGmsTmsTmsTmsGmsGmsCmsTm |
| 2984_as_MOE_L9 | sGmsAmsAm |
| NM_005249.5_2965- | TmsAmsAmsAmsTmsTmsTmsTmsAmsGbsTmsTmsTmsGmsGmsCmsTm |
| 2984_as_MOE_L10 | sGmsAmsAm |
| NM_005249.5_2965- | TmsAmsAmsAmsTmsTmsTmsTmsAmsGmsTbsTmsTmsGmsGmsCmsTm |
| 2984_as_MOE_L11 | sGmsAmsAm |
| NM_005249.5_2965- | TmsAmsAmsAmsTmsTmsTmsTmsAmsGmsTmsTbsTmsGmsGmsCmsTm |
| 2984_as_MOE_L12 | sGmsAmsAm |
| NM_005249.5_2965- | TmsAmsAmsAmsTmsTmsTmsTmsAmsGmsTmsTmsTbsGmsGmsCmsTm |
| 2984_as_MOE_L13 | sGmsAmsAm |
| NM_005249.5_2965- | TmsAmsAmsAmsTmsTmsTmsTmsAmsGmsTmsTmsTmsGbsGmsCmsTm |
| 2984_as_MOE_L14 | sGmsAmsAm |
| NM_005249.5_2965- | TmsAmsAmsAmsTmsTmsTmsTmsAmsGmsTmsTmsTmsGmsGbsCmsTm |
| 2984_as_MOE_L15 | sGmsAmsAm |
| NM_005249.5_2965- | TmsAmsAmsAmsTmsTmsTmsTmsAmsGmsTmsTmsTmsGmsGmsCbsTm |
| 2984_as_MOE_L16 | sGmsAmsAm |
| NM_005249.5_2965- | TmsAmsAmsAmsTmsTmsTmsTmsAmsGmsTmsTmsTmsGmsGmsCmsTb |
| 2984_as_MOE_L17 | sGmsAmsAm |
| NM_005249.5_2965- | TmsAmsAmsAmsTmsTmsTmsTmsAmsGmsTmsTmsTmsGmsGmsCmsTm |
| 2984_as_MOE_L18 | sGbsAmsAm |
| NM_005249.5_2965- | TmsAmsAmsAmsTmsTmsTmsTmsAmsGmsTmsTmsTmsGmsGmsCmsTm |
| 2984_as_MOE_L19 | sGmsAbsAm |
| NM_005249.5_2965- | TmsAmsAmsAmsTmsTmsTmsTmsAmsGmsTmsTmsTmsGmsGmsCmsTm |
| 2984_as_MOE_L20 | sGmsAmsAb |
| NM_005249.5_2965- | TbsAmsAmsAmsTmsTmsTmsTmsAmsGmsTmsTmsTmsGmsGmsCmsTm |
| 2984_as_MOE_L1_L20 | sGmsAmsAb |
| NM_005249.5_2965- | TmsAbsAmsAmsTmsTmsTmsTmsAmsGmsTmsTmsTmsGmsGmsCmsTm |
| 2984_as_MOE_L2_L19 | sGmsAbsAm |
| NM_005249.5_2965- | TmsAmsAbsAmsTmsTmsTmsTmsAmsGmsTmsTmsTmsGmsGmsCmsTm |
| 2984_as_MOE_L3_L18 | sGbsAmsAm |
| NM_005249.5_2965- | TmsAmsAmsAbsTmsTmsTmsTmsAmsGmsTmsTmsTmsGmsGmsCmsTb |
| 2984_as_MOE_L4_L17 | sGmsAmsAm |
| NM_005249.5_2965- | TmsAmsAmsAmsTbsTmsTmsTmsAmsGmsTmsTmsTmsGmsGmsCbsTm |
| 2984_as_MOE_L5_L16 | sGmsAmsAm |
| NM_005249.5_2965- | TmsAmsAmsAmsTmsTbsTmsTmsAmsGmsTmsTmsTmsGmsGbsCmsTm |
| 2984_as_MOE_L6_L15 | sGmsAmsAm |
| NM_005249.5_2965- | TmsAmsAmsAmsTmsTmsTbsTmsAmsGmsTmsTmsTmsGbsGmsCmsTm |
| 2984_as_MOE_L7_L14 | sGmsAmsAm |
| NM_005249.5_2965- | TmsAmsAmsAmsTmsTmsTmsTbsAmsGmsTmsTmsTbsGmsGmsCmsTm |
| 2984_as_MOE_L8_L13 | sGmsAmsAm |
| NM_005249.5_2965- | TmsAmsAmsAmsTmsTmsTmsTmsAbsGmsTmsTbsTmsGmsGmsCmsTm |
| 2984_as_MOE_L9_L12 | sGmsAmsAm |
| NM_005249.5_2965- | TmsAmsAmsAmsTmsTmsTmsTmsAmsGbsTbsTmsTmsGmsGmsCmsTm |
| 2984_as_MOE_L10_L11 | sGmsAmsAm |
| NM_005249.5_2965- | TmsAmAmsAmsTmsTmsTmsTmsAmsGmsTmsTmsTmsGmsGmsCmsTms |
| 2984_as_MOE_PO2 | GmsAmsAm |
| NM_005249.5_2965- | TmsAmsAmAmsTmsTmsTmsTmsAmsGmsTmsTmsTmsGmsGmsCmsTms |
| 2984_as_MOE_PO3 | GmsAmsAm |
| NM_005249.5_2965- | TmsAmsAmsAmTmsTmsTmsTmsAmsGmsTmsTmsTmsGmsGmsCmsTms |
| 2984_as_MOE_PO4 | GmsAmsAm |
| NM_005249.5_2965- | TmsAmsAmsAmsTmTmsTmsTmsAmsGmsTmsTmsTmsGmsGmsCmsTms |
| 2984_as_MOE_PO5 | GmsAmsAm |
| NM_005249.5_2965- | TmsAmsAmsAmsTmsTmTmsTmsAmsGmsTmsTmsTmsGmsGmsCmsTms |
| 2984_as_MOE_PO6 | GmsAmsAm |
| NM_005249.5_2965- | TmsAmsAmsAmsTmsTmsTmTmsAmsGmsTmsTmsTmsGmsGmsCmsTms |
| 2984_as_MOE_PO7 | GmsAmsAm |
| NM_005249.5_2965- | TmsAmsAmsAmsTmsTmsTmsTmAmsGmsTmsTmsTmsGmsGmsCmsTms |
| 2984_as_MOE_PO8 | GmsAmsAm |
| NM_005249.5_2965- | TmsAmsAmsAmsTmsTmsTmsTmsAmGmsTmsTmsTmsGmsGmsCmsTms |
| 2984_as_MOE_PO9 | GmsAmsAm |
| NM_005249.5_2965- | TmsAmsAmsAmsTmsTmsTmsTmsAmsGmTmsTmsTmsGmsGmsCmsTms |
| 2984_as_MOE_PO10 | GmsAmsAm |
| NM_005249.5_2965- | TmsAmsAmsAmsTmsTmsTmsTmsAmsGmsTmTmsTmsGmsGmsCmsTms |
| 2984_as_MOE_PO11 | GmsAmsAm |
| NM_005249.5_2965- | TmsAmsAmsAmsTmsTmsTmsTmsAmsGmsTmsTmTmsGmsGmsCmsTms |
| 2984_as_MOE_PO12 | GmsAmsAm |
| NM_005249.5_2965- | TmsAmsAmsAmsTmsTmsTmsTmsAmsGmsTmsTmsTmGmsGmsCmsTms |
| 2984_as_MOE_PO13 | GmsAmsAm |
| NM_005249.5_2965- | TmsAmsAmsAmsTmsTmsTmsTmsAmsGmsTmsTmsTmsGmGmsCmsTms |
| 2984_as_MOE_PO14 | GmsAmsAm |
| NM_005249.5_2965- | TmsAmsAmsAmsTmsTmsTmsTmsAmsGmsTmsTmsTmsGmsGmCmsTms |
| 2984_as_MOE_PO15 | GmsAmsAm |
| NM_005249.5_2965- | TmsAmsAmsAmsTmsTmsTmsTmsAmsGmsTmsTmsTmsGmsGmsCmTms |
| 2984_as_MOE_PO16 | GmsAmsAm |
| NM_005249.5_2965- | TmsAmsAmsAmsTmsTmsTmsTmsAmsGmsTmsTmsTmsGmsGmsCmsTm |
| 2984_as_MOE_PO17 | GmsAmsAm |
| NM_005249.5_2965- | TmsAmsAmsAmsTmsTmsTmsTmsAmsGmsTmsTmsTmsGmsGmsCmsTm |
| 2984_as_MOE_PO18 | sGmAmsAm |
| NM_005249.5_2965- | TmsAmAmsAmsTmsTmsTmsTmsAmsGmsTmsTmsTmsGmsGmsCmsTms |
| 2984_as_MOE_PO2_ | GmAmsAm |
| PO18 | |
| NM_005249.5_2965- | TmsAmsAmAmsTmsTmsTmsTmsAmsGmsTmsTmsTmsGmsGmsCmsTmG |
| 2984_as_MOE_PO3_ | msAmsAm |
| PO17 | |
| NM_005249.5_2965- | TmsAmsAmsAmTmsTmsTmsTmsAmsGmsTmsTmsTmsGmsGmsCmTmsG |
| 2984_as_MOE_PO4_ | msAmsAm |
| PO16 | |
| NM_005249.5_2965- | TmsAmsAmsAmsTmTmsTmsTmsAmsGmsTmsTmsTmsGmsGmCmsTmsG |
| 2984_as_MOE_PO5_ | msAmsAm |
| PO15 | |
| NM_005249.5_2965- | TmsAmsAmsAmsTmsTmTmsTmsAmsGmsTmsTmsTmsGmGmsCmsTmsG |
| 2984_as_MOE_PO6_ | msAmsAm |
| PO14 | |
| NM_005249.5_2965- | TmsAmsAmsAmsTmsTmsTmTmsAmsGmsTmsTmsTmGmsGmsCmsTmsG |
| 2984_as_MOE_PO7_ | msAmsAm |
| PO13 | |
| NM_005249.5_2965- | TmsAmsAmsAmsTmsTmsTmsTmAmsGmsTmsTmTmsGmsGmsCmsTmsG |
| 2984_as_MOE_PO8_ | msAmsAm |
| PO12 | |
| NM_005249.5_2965- | TmsAmsAmsAmsTmsTmsTmsTmsAmGmsTmTmsTmsGmsGmsCmsTmsG |
| 2984_as_MOE_PO9_ | msAmsAm |
| PO11 | |
| NM_005249.5_2965- | TmsAmsAmsAmsTmsTmsTmsTmsAmGmTmsTmsTmsGmsGmsCmsTmsG |
| 2984_as_MOE_PO9_ | msAmsAm |
| PO10 | |
| NM_005249.5_2965- | TmsAmsAmsAmsTmsTmsTmsTmsAmsGmTmTmsTmsGmsGmsCmsTmsG |
| 2984_as_MOE_PO10_ | msAmsAm |
| PO11 | |
| NM_005249.5_2965- | TmsAmAmsAmsTmsTmsTmsTmsAmsGmsTmsTmsTmsGmsGmsCmsTmG |
| 2984_as_MOE_PO2_ | msAmsAm |
| PO17 | |
| NM_005249.5_2965- | TmsAmsAmAmsTmsTmsTmsTmsAmsGmsTmsTmsTmsGmsGmsCmsTms |
| 2984_as_MOE_PO3_ | GmAmsAm |
| PO18 | |
| NM_005249.5_2965- | TmsAmsAmAmsTmsTmsTmsTmsAmsGmsTmsTmsTmsGmsGmsCmTmsG |
| 2984_as_MOE_PO3_ | msAmsAm |
| PO16 | |
| NM_005249.5_2965- | TmsAmsAmsAmTmsTmsTmsTmsAmsGmsTmsTmsTmsGmsGmsCmsTmG |
| 2984_as_MOE_PO4_ | msAmsAm |
| PO17 | |
| NM_005249.5_2965- | TmsAmsAmsAmTmsTmsTmsTmsAmsGmsTmsTmsTmsGmsGmCmsTmsG |
| 2984_as_MOE_PO4_ | msAmsAm |
| PO15 | |
| NM_005249.5_2965- | TmsAmsAmsAmsTmTmsTmsTmsAmsGmsTmsTmsTmsGmsGmsCmTmsG |
| 2984_as_MOE_PO5_ | msAmsAm |
| PO16 | |

TABLE 7
NM_005249.5 2062-2081 as MOE Modification Sequences

| Name | Sequence |
|---|---|
| NM_005249.5_2062- | AmsAmsAmsCmsGmsTmsAmsCmsAmsGmsAmsAmsAmsTmsGmsGmsGm |
| 2081_as_MOE | sAmsGmsGm |
| NM_005249.5_2062- | AbsAmsAmsCmsGmsTmsAmsCmsAmsGmsAmsAmsAmsTmsGmsGmsGm |
| 2081_as_MOE_L1 | sAmsGmsGm |
| NM_005249.5_2062- | AmsAbsAmsCmsGmsTmsAmsCmsAmsGmsAmsAmsAmsTmsGmsGmsGm |
| 2081_as_MOE_L2 | sAmsGmsGm |
| NM_005249.5_2062- | AmsAmsAbsCmsGmsTmsAmsCmsAmsGmsAmsAmsAmsTmsGmsGmsGm |
| 2081_as_MOE_L3 | sAmsGmsGm |
| NM_005249.5_2062- | AmsAmsAmsCbsGmsTmsAmsCmsAmsGmsAmsAmsAmsTmsGmsGmsGm |
| 2081_as_MOE_L4 | sAmsGmsGm |
| NM_005249.5_2062- | AmsAmsAmsCmsGbsTmsAmsCmsAmsGmsAmsAmsAmsTmsGmsGmsGm |
| 2081_as_MOE_L5 | sAmsGmsGm |
| NM_005249.5_2062- | AmsAmsAmsCmsGmsTbsAmsCmsAmsGmsAmsAmsAmsTmsGmsGmsGm |
| 2081_as_MOE_L6 | sAmsGmsGm |
| NM_005249.5_2062- | AmsAmsAmsCmsGmsTmsAbsCmsAmsGmsAmsAmsAmsTmsGmsGmsGm |
| 2081_as_MOE_L7 | sAmsGmsGm |
| NM_005249.5_2062- | AmsAmsAmsCmsGmsTmsAmsCbsAmsGmsAmsAmsAmsTmsGmsGmsGm |
| 2081_as_MOE_L8 | sAmsGmsGm |
| NM_005249.5_2062- | AmsAmsAmsCmsGmsTmsAmsCmsAbsGmsAmsAmsAmsTmsGmsGmsGm |
| 2081_as_MOE_L9 | sAmsGmsGm |
| NM_005249.5_2062- | AmsAmsAmsCmsGmsTmsAmsCmsAmsGbsAmsAmsAmsTmsGmsGmsGm |
| 2081_as_MOE_L10 | sAmsGmsGm |
| NM_005249.5_2062- | AmsAmsAmsCmsGmsTmsAmsCmsAmsGmsAbsAmsAmsTmsGmsGmsGm |
| 2081_as_MOE_L11 | sAmsGmsGm |
| NM_005249.5_2062- | AmsAmsAmsCmsGmsTmsAmsCmsAmsGmsAmsAbsAmsTmsGmsGmsGm |
| 2081_as_MOE_L12 | sAmsGmsGm |
| NM_005249.5_2062- | AmsAmsAmsCmsGmsTmsAmsCmsAmsGmsAmsAmsAbsTmsGmsGmsGm |
| 2081_as_MOE_L13 | sAmsGmsGm |
| NM_005249.5_2062- | AmsAmsAmsCmsGmsTmsAmsCmsAmsGmsAmsAmsAmsTbsGmsGmsGm |
| 2081_as_MOE_L14 | sAmsGmsGm |
| NM_005249.5_2062- | AmsAmsAmsCmsGmsTmsAmsCmsAmsGmsAmsAmsAmsTmsGbsGmsGm |
| 2081_as_MOE_L15 | sAmsGmsGm |
| NM_005249.5_2062- | AmsAmsAmsCmsGmsTmsAmsCmsAmsGmsAmsAmsAmsTmsGmsGbsGm |
| 2081_as_MOE_L16 | sAmsGmsGm |
| NM_005249.5_2062- | AmsAmsAmsCmsGmsTmsAmsCmsAmsGmsAmsAmsAmsTmsGmsGmsGb |
| 2081_as_MOE_L17 | sAmsGmsGm |
| NM_005249.5_2062- | AmsAmsAmsCmsGmsTmsAmsCmsAmsGmsAmsAmsAmsTmsGmsGmsGm |
| 2081_as_MOE_L18 | sAbsGmsGm |
| NM_005249.5_2062- | AmsAmsAmsCmsGmsTmsAmsCmsAmsGmsAmsAmsAmsTmsGmsGmsGm |
| 2081_as_MOE_L19 | sAmsGbsGm |
| NM_005249.5_2062- | AmsAmsAmsCmsGmsTmsAmsCmsAmsGmsAmsAmsAmsTmsGmsGmsGm |
| 2081_as_MOE_L20 | sAmsGmsGb |
| NM_005249.5_2062- | AbsAmsAmsCmsGmsTmsAmsCmsAmsGmsAmsAmsAmsTmsGmsGmsGm |
| 2081_as_MOE_L1_L20 | sAmsGmsGb |
| NM_005249.5_2062- | AmsAbsAmsCmsGmsTmsAmsCmsAmsGmsAmsAmsAmsTmsGmsGmsGm |
| 2081_as_MOE_L2_L19 | sAmsGbsGm |
| NM_005249.5_2062- | AmsAmsAbsCmsGmsTmsAmsCmsAmsGmsAmsAmsAmsTmsGmsGmsGm |
| 2081_as_MOE_L3_L18 | sAbsGmsGm |
| NM_005249.5_2062- | AmsAmsAmsCbsGmsTmsAmsCmsAmsGmsAmsAmsAmsTmsGmsGmsGb |
| 2081_as_MOE_L4_L17 | sAmsGmsGm |
| NM_005249.5_2062- | AmsAmsAmsCmsGbsTmsAmsCmsAmsGmsAmsAmsAmsTmsGmsGbsGm |
| 2081_as_MOE_L5_L16 | sAmsGmsGm |
| NM_005249.5_2062- | AmsAmsAmsCmsGmsTbsAmsCmsAmsGmsAmsAmsAmsTmsGbsGmsGm |
| 2081_as_MOE_L6_L15 | sAmsGmsGm |
| NM_005249.5_2062- | AmsAmsAmsCmsGmsTmsAbsCmsAmsGmsAmsAmsAmsTbsGmsGmsGm |
| 2081_as_MOE_L7_L14 | sAmsGmsGm |
| NM_005249.5_2062- | AmsAmsAmsCmsGmsTmsAmsCbsAmsGmsAmsAmsAbsTmsGmsGmsGm |
| 2081_as_MOE_L8_L13 | sAmsGmsGm |
| NM_005249.5_2062- | AmsAmsAmsCmsGmsTmsAmsCmsAbsGmsAmsAbsAmsTmsGmsGmsGm |
| 2081_as_MOE_L9_L12 | sAmsGmsGm |
| NM_005249.5_2062- | AmsAmsAmsCmsGmsTmsAmsCmsAmsGbsAbsAmsAmsTmsGmsGmsGm |
| 2081_as_MOE_L10_L11 | sAmsGmsGm |
| NM_005249.5_2062- | AmsAmAmsCmsGmsTmsAmsCmsAmsGmsAmsAmsAmsTmsGmsGmsGms |
| 2081_as_MOE_PO2 | AmsGmsGm |
| NM_005249.5_2062- | AmsAmsAmCmsGmsTmsAmsCmsAmsGmsAmsAmsAmsTmsGmsGmsGms |
| 2081_as_MOE_PO3 | AmsGmsGm |
| NM_005249.5_2062- | AmsAmsAmsCmGmsTmsAmsCmsAmsGmsAmsAmsAmsTmsGmsGmsGms |
| 2081_as_MOE_PO4 | AmsGmsGm |
| NM_005249.5_2062- | AmsAmsAmsCmsGmTmsAmsCmsAmsGmsAmsAmsAmsTmsGmsGmsGms |
| 2081_as_MOE_PO5 | AmsGmsGm |
| NM_005249.5_2062- | AmsAmsAmsCmsGmsTmAmsCmsAmsGmsAmsAmsAmsTmsGmsGmsGms |
| 2081_as_MOE_PO6 | AmsGmsGm |
| NM_005249.5_2062- | AmsAmsAmsCmsGmsTmsAmCmsAmsGmsAmsAmsAmsTmsGmsGmsGms |
| 2081_as_MOE_PO7 | AmsGmsGm |
| NM_005249.5_2062- | AmsAmsAmsCmsGmsTmsAmsCmAmsGmsAmsAmsAmsTmsGmsGmsGms |
| 2081_as_MOE_PO8 | AmsGmsGm |
| NM_005249.5_2062- | AmsAmsAmsCmsGmsTmsAmsCmsAmGmsAmsAmsAmsTmsGmsGmsGms |
| 2081_as_MOE_PO9 | AmsGmsGm |
| NM_005249.5_2062- | AmsAmsAmsCmsGmsTmsAmsCmsAmsGmAmsAmsAmsTmsGmsGmsGms |
| 2081_as_MOE_PO10 | AmsGmsGm |
| NM_005249.5_2062- | AmsAmsAmsCmsGmsTmsAmsCmsAmsGmsAmAmsAmsTmsGmsGmsGms |
| 2081_as_MOE_PO11 | AmsGmsGm |
| NM_005249.5_2062- | AmsAmsAmsCmsGmsTmsAmsCmsAmsGmsAmsAmAmsTmsGmsGmsGms |
| 2081_as_MOE_PO12 | AmsGmsGm |
| NM_005249.5_2062- | AmsAmsAmsCmsGmsTmsAmsCmsAmsGmsAmsAmsAmTmsGmsGmsGms |
| 2081_as_MOE_PO13 | AmsGmsGm |
| NM_005249.5_2062- | AmsAmsAmsCmsGmsTmsAmsCmsAmsGmsAmsAmsAmsTmGmsGmsGms |
| 2081_as_MOE_PO14 | AmsGmsGm |
| NM_005249.5_2062- | AmsAmsAmsCmsGmsTmsAmsCmsAmsGmsAmsAmsAmsTmsGmGmsGms |
| 2081_as_MOE_PO15 | AmsGmsGm |
| NM_005249.5_2062- | AmsAmsAmsCmsGmsTmsAmsCmsAmsGmsAmsAmsAmsTmsGmsGmGms |
| 2081_as_MOE_PO16 | AmsGmsGm |
| NM_005249.5_2062- | AmsAmsAmsCmsGmsTmsAmsCmsAmsGmsAmsAmsAmsTmsGmsGmsGm |
| 2081_as_MOE_PO17 | AmsGmsGm |
| NM_005249.5_2062- | AmsAmsAmsCmsGmsTmsAmsCmsAmsGmsAmsAmsAmsTmsGmsGmsGm |
| 2081_as_MOE_PO18 | sAmGmsGm |
| NM_005249.5_2062- | AmsAmAmsCmsGmsTmsAmsCmsAmsGmsAmsAmsAmsTmsGmsGmsGms |
| 2081_as_MOE_PO2_ | AmGmsGm |
| PO18 | |
| NM_005249.5_2062- | AmsAmsAmCmsGmsTmsAmsCmsAmsGmsAmsAmsAmsTmsGmsGmsGmA |
| 2081_as_MOE_PO3_ | msGmsGm |
| PO17 | |
| NM_005249.5_2062- | AmsAmsAmsCmGmsTmsAmsCmsAmsGmsAmsAmsAmsTmsGmsGmGmsA |
| 2081_as_MOE_PO4_ | msGmsGm |
| PO16 | |
| NM_005249.5_2062- | AmsAmsAmsCmsGmTmsAmsCmsAmsGmsAmsAmsAmsTmsGmGmsGmsA |
| 2081_as_MOE_PO5_ | msGmsGm |
| PO15 | |
| NM_005249.5_2062- | AmsAmsAmsCmsGmsTmAmsCmsAmsGmsAmsAmsAmsTmGmsGmsGmsA |
| 2081_as_MOE_PO6_ | msGmsGm |
| PO14 | |
| NM_005249.5_2062- | AmsAmsAmsCmsGmsTmsAmCmsAmsGmsAmsAmsAmTmsGmsGmsGmsA |
| 2081_as_MOE_PO7_ | msGmsGm |
| PO13 | |
| NM_005249.5_2062- | AmsAmsAmsCmsGmsTmsAmsCmAmsGmsAmsAmAmsTmsGmsGmsGmsA |
| 2081_as_MOE_PO8_ | msGmsGm |
| PO12 | |
| NM_005249.5_2062- | AmsAmsAmsCmsGmsTmsAmsCmsAmGmsAmAmsAmsTmsGmsGmsGmsA |
| 2081_as_MOE_PO9_ | msGmsGm |
| PO11 | |
| NM_005249.5_2062- | AmsAmsAmsCmsGmsTmsAmsCmsAmGmAmsAmsAmsTmsGmsGmsGmsA |
| 2081_as_MOE_PO9_ | msGmsGm |
| PO10 | |
| NM_005249.5_2062- | AmsAmsAmsCmsGmsTmsAmsCmsAmsGmAmAmsAmsTmsGmsGmsGmsA |
| 2081_as_MOE_PO10_ | msGmsGm |
| PO11 | |
| NM_005249.5_2062- | AmsAmAmsCmsGmsTmsAmsCmsAmsGmsAmsAmsAmsTmsGmsGmsGmA |
| 2081_as_MOE_PO2_ | msGmsGm |
| PO17 | |
| NM_005249.5_2062- | AmsAmsAmCmsGmsTmsAmsCmsAmsGmsAmsAmsAmsTmsGmsGmsGms |
| 2081_as_MOE_PO3_ | AmGmsGm |
| PO18 | |
| NM_005249.5_2062- | AmsAmsAmCmsGmsTmsAmsCmsAmsGmsAmsAmsAmsTmsGmsGmGmsA |
| 2081_as_MOE_PO3_ | msGmsGm |
| PO16 | |
| NM_005249.5_2062- | AmsAmsAmsCmGmsTmsAmsCmsAmsGmsAmsAmsAmsTmsGmsGmsGmA |
| 2081_as_MOE_PO4_ | msGmsGm |
| PO17 | |
| NM_005249.5_2062- | AmsAmsAmsCmGmsTmsAmsCmsAmsGmsAmsAmsAmsTmsGmGmsGmsA |
| 2081_as_MOE_PO4_ | msGmsGm |
| PO15 | |
| NM_005249.5_2062- | AmsAmsAmsCmsGmTmsAmsCmsAmsGmsAmsAmsAmsTmsGmsGmGmsA |
| 2081_as_MOE_PO5_ | msGmsGm |
| PO16 | |

TABLE 8
NM_005249.5 2061-2080 as MOE Modification Sequences

| Name | Sequence |
|---|---|
| NM_005249.5_2061- | AmsAmsCmsGmsTmsAmsCmsAmsGmsAmsAmsAmsTmsGmsGmsGmsAm |
| 2080_as_MOE | sGmsGmsGm |
| NM_005249.5_2061- | AbsAmsCmsGmsTmsAmsCmsAmsGmsAmsAmsAmsTmsGmsGmsGmsAm |
| 2080_as_MOE_L1 | SGmsGmsGm |
| NM_005249.5_2061- | AmsAbsCmsGmsTmsAmsCmsAmsGmsAmsAmsAmsTmsGmsGmsGmsAm |
| 2080_as_MOE_L2 | sGmsGmsGm |
| NM_005249.5_2061- | AmsAmsCbsGmsTmsAmsCmsAmsGmsAmsAmsAmsTmsGmsGmsGmsAm |
| 2080_as_MOE_L3 | sGmsGmsGm |
| NM_005249.5_2061- | AmsAmsCmsGbsTmsAmsCmsAmsGmsAmsAmsAmsTmsGmsGmsGmsAm |
| 2080_as_MOE_L4 | sGmsGmsGm |
| NM_005249.5_2061- | AmsAmsCmsGmsTbsAmsCmsAmsGmsAmsAmsAmsTmsGmsGmsGmsAm |
| 2080_as_MOE_L5 | sGmsGmsGm |
| NM_005249.5_2061- | AmsAmsCmsGmsTmsAbsCmsAmsGmsAmsAmsAmsTmsGmsGmsGmsAm |
| 2080_as_MOE_L6 | sGmsGmsGm |
| NM_005249.5_2061- | AmsAmsCmsGmsTmsAmsCbsAmsGmsAmsAmsAmsTmsGmsGmsGmsAm |
| 2080_as_MOE_L7 | sGmsGmsGm |
| NM_005249.5_2061- | AmsAmsCmsGmsTmsAmsCmsAbsGmsAmsAmsAmsTmsGmsGmsGmsAm |
| 2080_as_MOE_L8 | sGmsGmsGm |
| NM_005249.5_2061- | AmsAmsCmsGmsTmsAmsCmsAmsGbsAmsAmsAmsTmsGmsGmsGmsAm |
| 2080_as_MOE_L9 | sGmsGmsGm |
| NM_005249.5_2061- | AmsAmsCmsGmsTmsAmsCmsAmsGmsAbsAmsAmsTmsGmsGmsGmsAm |
| 2080_as_MOE_L10 | sGmsGmsGm |
| NM_005249.5_2061- | AmsAmsCmsGmsTmsAmsCmsAmsGmsAmsAbsAmsTmsGmsGmsGmsAm |
| 2080_as_MOE_L11 | sGmsGmsGm |
| NM_005249.5_2061- | AmsAmsCmsGmsTmsAmsCmsAmsGmsAmsAmsAbsTmsGmsGmsGmsAm |
| 2080_as_MOE_L12 | sGmsGmsGm |
| NM_005249.5_2061- | AmsAmsCmsGmsTmsAmsCmsAmsGmsAmsAmsAmsTbsGmsGmsGmsAm |
| 2080_as_MOE_L13 | sGmsGmsGm |
| NM_005249.5_2061- | AmsAmsCmsGmsTmsAmsCmsAmsGmsAmsAmsAmsTmsGbsGmsGmsAm |
| 2080_as_MOE_L14 | sGmsGmsGm |
| NM_005249.5_2061- | AmsAmsCmsGmsTmsAmsCmsAmsGmsAmsAmsAmsTmsGmsGbsGmsAm |
| 2080_as_MOE_L15 | sGmsGmsGm |
| NM_005249.5_2061- | AmsAmsCmsGmsTmsAmsCmsAmsGmsAmsAmsAmsTmsGmsGmsGbsAm |
| 2080_as_MOE_L16 | sGmsGmsGm |
| NM_005249.5_2061- | AmsAmsCmsGmsTmsAmsCmsAmsGmsAmsAmsAmsTmsGmsGmsGmsAb |
| 2080_as_MOE_L17 | sGmsGmsGm |
| NM_005249.5_2061- | AmsAmsCmsGmsTmsAmsCmsAmsGmsAmsAmsAmsTmsGmsGmsGmsAm |
| 2080_as_MOE_L18 | sGbsGmsGm |
| NM_005249.5_2061- | AmsAmsCmsGmsTmsAmsCmsAmsGmsAmsAmsAmsTmsGmsGmsGmsAm |
| 2080_as_MOE_L19 | sGmsGbsGm |
| NM_005249.5_2061- | AmsAmsCmsGmsTmsAmsCmsAmsGmsAmsAmsAmsTmsGmsGmsGmsAm |
| 2080_as_MOE_L20 | sGmsGmsGb |
| NM_005249.5_2061- | AbsAmsCmsGmsTmsAmsCmsAmsGmsAmsAmsAmsTmsGmsGmsGmsAm |
| 2080_as_MOE_L1_L20 | sGmsGmsGb |
| NM_005249.5_2061- | AmsAbsCmsGmsTmsAmsCmsAmsGmsAmsAmsAmsTmsGmsGmsGmsAm |
| 2080_as_MOE_L2_L19 | sGmsGbsGm |
| NM_005249.5_2061- | AmsAmsCbsGmsTmsAmsCmsAmsGmsAmsAmsAmsTmsGmsGmsGmsAm |
| 2080_as_MOE_L3_L18 | sGbsGmsGm |
| NM_005249.5_2061- | AmsAmsCmsGbsTmsAmsCmsAmsGmsAmsAmsAmsTmsGmsGmsGmsAb |
| 2080_as_MOE_L4_L17 | sGmsGmsGm |
| NM_005249.5_2061- | AmsAmsCmsGmsTbsAmsCmsAmsGmsAmsAmsAmsTmsGmsGmsGbsAm |
| 2080_as_MOE_L5_L16 | sGmsGmsGm |
| NM_005249.5_2061- | AmsAmsCmsGmsTmsAbsCmsAmsGmsAmsAmsAmsTmsGmsGbsGmsAm |
| 2080_as_MOE_L6_L15 | sGmsGmsGm |
| NM_005249.5_2061- | AmsAmsCmsGmsTmsAmsCbsAmsGmsAmsAmsAmsTmsGbsGmsGmsAm |
| 2080_as_MOE_L7_L14 | sGmsGmsGm |
| NM_005249.5_2061- | AmsAmsCmsGmsTmsAmsCmsAbsGmsAmsAmsAmsTbsGmsGmsGmsAm |
| 2080_as_MOE_L8_L13 | sGmsGmsGm |
| NM_005249.5_2061- | AmsAmsCmsGmsTmsAmsCmsAmsGbsAmsAmsAbsTmsGmsGmsGmsAm |
| 2080_as_MOE_L9_L12 | sGmsGmsGm |
| NM_005249.5_2061- | AmsAmsCmsGmsTmsAmsCmsAmsGmsAbsAbsAmsTmsGmsGmsGmsAm |
| 2080_as_MOE_L10_L11 | sGmsGmsGm |
| NM_005249.5_2061- | AmsAmCmsGmsTmsAmsCmsAmsGmsAmsAmsAmsTmsGmsGmsGmsAms |
| 2080_as_MOE_PO2 | GmsGmsGm |
| NM_005249.5_2061- | AmsAmsCmGmsTmsAmsCmsAmsGmsAmsAmsAmsTmsGmsGmsGmsAms |
| 2080_as_MOE_PO3 | GmsGmsGm |
| NM_005249.5_2061- | AmsAmsCmsGmTmsAmsCmsAmsGmsAmsAmsAmsTmsGmsGmsGmsAms |
| 2080_as_MOE_PO4 | GmsGmsGm |
| NM_005249.5_2061- | AmsAmsCmsGmsTmAmsCmsAmsGmsAmsAmsAmsTmsGmsGmsGmsAms |
| 2080_as_MOE_PO5 | GmsGmsGm |
| NM_005249.5_2061- | AmsAmsCmsGmsTmsAmCmsAmsGmsAmsAmsAmsTmsGmsGmsGmsAms |
| 2080_as_MOE_PO6 | GmsGmsGm |
| NM_005249.5_2061- | AmsAmsCmsGmsTmsAmsCmAmsGmsAmsAmsAmsTmsGmsGmsGmsAms |
| 2080_as_MOE_PO7 | GmsGmsGm |
| NM_005249.5_2061- | AmsAmsCmsGmsTmsAmsCmsAmGmsAmsAmsAmsTmsGmsGmsGmsAms |
| 2080_as_MOE_PO8 | GmsGmsGm |
| NM_005249.5_2061- | AmsAmsCmsGmsTmsAmsCmsAmsGmAmsAmsAmsTmsGmsGmsGmsAms |
| 2080_as_MOE_PO9 | GmsGmsGm |
| NM_005249.5_2061- | AmsAmsCmsGmsTmsAmsCmsAmsGmsAmAmsAmsTmsGmsGmsGmsAms |
| 2080_as_MOE_PO10 | GmsGmsGm |
| NM_005249.5_2061- | AmsAmsCmsGmsTmsAmsCmsAmsGmsAmsAmAmsTmsGmsGmsGmsAms |
| 2080_as_MOE_PO11 | GmsGmsGm |
| NM_005249.5_2061- | AmsAmsCmsGmsTmsAmsCmsAmsGmsAmsAmsAmTmsGmsGmsGmsAms |
| 2080_as_MOE_PO12 | GmsGmsGm |
| NM_005249.5_2061- | AmsAmsCmsGmsTmsAmsCmsAmsGmsAmsAmsAmsTmGmsGmsGmsAms |
| 2080_as_MOE_PO13 | GmsGmsGm |
| NM_005249.5_2061- | AmsAmsCmsGmsTmsAmsCmsAmsGmsAmsAmsAmsTmsGmGmsGmsAms |
| 2080_as_MOE_PO14 | GmsGmsGm |
| NM_005249.5_2061- | AmsAmsCmsGmsTmsAmsCmsAmsGmsAmsAmsAmsTmsGmsGmGmsAms |
| 2080_as_MOE_PO15 | GmsGmsGm |
| NM_005249.5_2061- | AmsAmsCmsGmsTmsAmsCmsAmsGmsAmsAmsAmsTmsGmsGmsGmAms |
| 2080_as_MOE_PO16 | GmsGmsGm |
| NM_005249.5_2061- | AmsAmsCmsGmsTmsAmsCmsAmsGmsAmsAmsAmsTmsGmsGmsGmsAm |
| 2080_as_MOE_PO17 | GmsGmsGm |
| NM_005249.5_2061- | AmsAmsCmsGmsTmsAmsCmsAmsGmsAmsAmsAmsTmsGmsGmsGmsAm |
| 2080_as_MOE_PO18 | sGmGmsGm |
| NM_005249.5_2061- | AmsAmCmsGmsTmsAmsCmsAmsGmsAmsAmsAmsTmsGmsGmsGmsAms |
| 2080_as_MOE_PO2_ | GmGmsGm |
| PO18 | |
| NM_005249.5_2061- | AmsAmsCmGmsTmsAmsCmsAmsGmsAmsAmsAmsTmsGmsGmsGmsAmG |
| 2080_as_MOE_PO3_ | msGmsGm |
| PO17 | |
| NM_005249.5_2061- | AmsAmsCmsGmTmsAmsCmsAmsGmsAmsAmsAmsTmsGmsGmsGmAmsG |
| 2080_as_MOE_PO4_ | msGmsGm |
| PO16 | |
| NM_005249.5_2061- | AmsAmsCmsGmsTmAmsCmsAmsGmsAmsAmsAmsTmsGmsGmGmsAmsG |
| 2080_as_MOE_PO5_ | msGmsGm |
| PO15 | |
| NM_005249.5_2061- | AmsAmsCmsGmsTmsAmCmsAmsGmsAmsAmsAmsTmsGmGmsGmsAmsG |
| 2080_as_MOE_PO6_ | msGmsGm |
| PO14 | |
| NM_005249.5_2061- | AmsAmsCmsGmsTmsAmsCmAmsGmsAmsAmsAmsTmGmsGmsGmsAmsG |
| 2080_as_MOE_PO7_ | msGmsGm |
| PO13 | |
| NM_005249.5_2061- | AmsAmsCmsGmsTmsAmsCmsAmGmsAmsAmsAmTmsGmsGmsGmsAmsG |
| 2080_as_MOE_PO8_ | msGmsGm |
| PO12 | |
| NM_005249.5_2061- | AmsAmsCmsGmsTmsAmsCmsAmsGmAmsAmAmsTmsGmsGmsGmsAmsG |
| 2080_as_MOE_PO9_ | msGmsGm |
| PO11 | |
| NM_005249.5_2061- | AmsAmsCmsGmsTmsAmsCmsAmsGmAmAmsAmsTmsGmsGmsGmsAmsG |
| 2080_as_MOE_PO9_ | msGmsGm |
| PO10 | |
| NM_005249.5_2061- | AmsAmsCmsGmsTmsAmsCmsAmsGmsAmAmAmsTmsGmsGmsGmsAmsG |
| 2080_as_MOE_PO10_ | msGmsGm |
| PO11 | |
| NM_005249.5_2061- | AmsAmCmsGmsTmsAmsCmsAmsGmsAmsAmsAmsTmsGmsGmsGmsAmG |
| 2080_as_MOE_PO2_ | msGmsGm |
| PO17 | |
| NM_005249.5_2061- | AmsAmsCmGmsTmsAmsCmsAmsGmsAmsAmsAmsTmsGmsGmsGmsAms |
| 2080_as_MOE_PO3_ | GmGmsGm |
| PO18 | |
| NM_005249.5_2061- | AmsAmsCmGmsTmsAmsCmsAmsGmsAmsAmsAmsTmsGmsGmsGmAmsG |
| 2080_as_MOE_PO3_ | msGmsGm |
| PO16 | |
| NM_005249.5_2061- | AmsAmsCmsGmTmsAmsCmsAmsGmsAmsAmsAmsTmsGmsGmsGmsAmG |
| 2080_as_MOE_PO4_ | msGmsGm |
| PO17 | |
| NM_005249.5_2061- | AmsAmsCmsGmTmsAmsCmsAmsGmsAmsAmsAmsTmsGmsGmGmsAmsG |
| 2080_as_MOE_PO4_ | msGmsGm |
| PO15 | |
| NM_005249.5_2061- | AmsAmsCmsGmsTmAmsCmsAmsGmsAmsAmsAmsTmsGmsGmsGmAmsG |
| 2080_as_MOE_PO5_ | msGmsGm |
| PO16 | |

### Transfection of ASOs and FOXG1 Quantification:

### Modulation of FOXG1 Expression by ASOs:

Table 9 shows the result on FOXG1 expression in HEK293 cells associated with transfection with modified ASOs based on the NM_005249.5_2965-2984_as_MOE sequence. Transfection was performed with ASOs at concentrations of 50 nM and 5 nM. After 24h of incubation with ASOs, medium was removed, the cells were lysed, and QuantiGene detection was used to determine FOXG1 mRNA expression in cells lysates.

Individual expression values were scaled relative to the GAPDH housekeeping gene, then all normalized to control (mock transfected cell mean value and RLuc-transfected cells). Modulation of GAPDH itself was used as a proxy for cell health, and only oligos that showed <25% reduction in GAPDH (along with FOXG1 upregulation) were selected for follow up.

| | % FOXG1 mRNA relative to GAPDH | | | |
|---|---|---|---|---|
| | Mean | | SD | |
| Name | 50 nM | 5 nM | 50 nM | 5 nM |
| NM_005249.5_2965- | 96.30 | 104.36 | 13.31 | 34.57 |
| 2984_as_MOE | | | | |
| NM_005249.5_2965- | 94.57 | 103.24 | 12.77 | 21.69 |
| 2984_as_MOE_L1 | | | | |
| NM_005249.5_2965- | 174.57 | 104.63 | 24.62 | 17.13 |
| 2984_as_MOE_L2 | | | | |
| NM_005249.5_2965- | 305.57 | 138.77 | 205.37 | 70.35 |
| 2984_as_MOE_L3 | | | | |
| NM_005249.5_2965- | 165.49 | 111.53 | 19.26 | 48.51 |
| 2984_as_MOE_L4 | | | | |
| NM_005249.5_2965- | 155.23 | 106.55 | 7.51 | 11.98 |
| 2984_as_MOE_L5 | | | | |
| NM_005249.5_2965- | 172.94 | 86.23 | 7.15 | 26.72 |
| 2984_as_MOE_L6 | | | | |
| NM_005249.5_2965- | 153.41 | 85.48 | 10.89 | 29.93 |
| 2984_as_MOE_L7 | | | | |
| NM_005249.5_2965- | 171.75 | 99.47 | 21.06 | 18.54 |
| 2984_as_MOE_L8 | | | | |
| NM_005249.5_2965- | 144.31 | 94.13 | 21.11 | 29.20 |
| 2984_as_MOE_L9 | | | | |
| NM_005249.5_2965- | 81.40 | 78.98 | 23.63 | 21.64 |
| 2984_as_MOE_L10 | | | | |
| NM_005249.5_2965- | 106.14 | 93.46 | 58.38 | 32.33 |
| 2984_as_MOE_L11 | | | | |
| NM_005249.5_2965- | 107.28 | 84.56 | 15.52 | 39.55 |
| 2984_as_MOE_L12 | | | | |
| NM_005249.5_2965- | 102.37 | 118.07 | 26.63 | 23.68 |
| 2984_as_MOE_L13 | | | | |
| NM_005249.5_2965- | 105.37 | 121.87 | 29.34 | 35.00 |
| 2984_as_MOE_L14 | | | | |
| NM_005249.5_2965- | 80.31 | 110.72 | 47.12 | 18.43 |
| 2984_as_MOE_L15 | | | | |
| NM_005249.5_2965- | 92.40 | 111.04 | 49.21 | 20.32 |
| 2984_as_MOE_L16 | | | | |
| NM_005249.5_2965- | 119.37 | 104.93 | 51.27 | 21.60 |
| 2984_as_MOE_L17 | | | | |
| NM_005249.5_2965- | 101.65 | 111.92 | 21.19 | 20.77 |
| 2984_as_MOE_L18 | | | | |
| NM_005249.5_2965- | 77.57 | 109.01 | 14.71 | 32.11 |
| 2984_as_MOE_L19 | | | | |
| NM_005249.5_2965- | 126.25 | 93.80 | 25.61 | 17.73 |
| 2984_as_MOE_L20 | | | | |
| NM_005249.5_2965- | 87.70 | 90.26 | 32.15 | 15.77 |
| 2984_as_MOE_L1_L20 | | | | |
| NM_005249.5_2965- | 119.43 | 91.99 | 32.26 | 11.46 |
| 2984_as_MOE_L2_L19 | | | | |
| NM_005249.5_2965- | 97.70 | 69.63 | 23.29 | 11.22 |
| 2984_as_MOE_L3_L18 | | | | |
| NM_005249.5_2965- | 107.44 | 72.44 | 24.31 | 15.88 |
| 2984_as_MOE_L4_L17 | | | | |
| NM_005249.5_2965- | 88.77 | 63.25 | 22.45 | 10.34 |
| 2984_as_MOE_L5_L16 | | | | |
| NM_005249.5_2965- | 91.91 | 65.92 | 17.80 | 11.22 |
| 2984_as_MOE_L6_L15 | | | | |
| NM_005249.5_2965- | 103.07 | 61.81 | 26.97 | 12.12 |
| 2984_as_MOE_L7_L14 | | | | |
| NM_005249.5_2965- | 130.21 | 112.51 | 37.31 | 85.42 |
| 2984_as_MOE_L8_L13 | | | | |
| NM_005249.5_2965- | 104.57 | 88.19 | 18.43 | 9.47 |
| 2984_as_MOE_L9_L12 | | | | |
| NM_005249.5_2965- | 90.16 | 65.04 | 9.46 | 12.99 |
| 2984_as_MOE_L10_L11 | | | | |
| NM_005249.5_2965- | 44.67 | 78.29 | 18.76 | 15.17 |
| 2984_as_MOE_PO2 | | | | |
| NM_005249.5_2965- | 58.40 | 70.31 | 12.11 | 11.89 |
| 2984_as_MOE_PO3 | | | | |
| NM_005249.5_2965- | 66.04 | 80.27 | 16.76 | 13.64 |
| 2984_as_MOE_PO4 | | | | |
| NM_005249.5_2965- | 50.91 | 68.65 | 2.46 | 5.38 |
| 2984_as_MOE_PO5 | | | | |
| NM_005249.5_2965- | 64.91 | 85.92 | 30.36 | 36.54 |
| 2984_as_MOE_PO6 | | | | |
| NM_005249.5_2965- | 51.60 | 74.68 | 11.17 | 21.09 |
| 2984_as_MOE_PO7 | | | | |
| NM_005249.5_2965- | 45.81 | 71.95 | 9.08 | 10.41 |
| 2984_as_MOE_PO8 | | | | |
| NM_005249.5_2965- | 65.85 | 60.25 | 23.93 | 14.03 |
| 2984_as_MOE_PO9 | | | | |
| NM_005249.5_2965- | 50.10 | 79.62 | 11.38 | 8.84 |
| 2984_as_MOE_PO10 | | | | |
| NM_005249.5_2965- | 87.67 | 110.25 | 25.37 | 17.73 |
| 2984_as_MOE_PO11 | | | | |
| NM_005249.5_2965- | 104.43 | 83.65 | 54.51 | 13.37 |
| 2984_as_MOE_PO12 | | | | |
| NM_005249.5_2965- | 101.41 | 104.72 | 23.02 | 22.32 |
| 2984_as_MOE_PO13 | | | | |
| NM_005249.5_2965- | 109.78 | 110.49 | 46.35 | 20.57 |
| 2984_as_MOE_PO14 | | | | |
| NM_005249.5_2965- | 108.32 | 98.27 | 18.15 | 15.32 |
| 2984_as_MOE_PO15 | | | | |
| NM_005249.5_2965- | 107.77 | 94.33 | 30.26 | 22.66 |
| 2984_as_MOE_PO16 | | | | |
| NM_005249.5_2965- | 92.39 | 96.53 | 18.47 | 29.93 |
| 2984_as_MOE_PO17 | | | | |
| NM_005249.5_2965- | 100.93 | 107.18 | 16.59 | 20.93 |
| 2984_as_MOE_PO18 | | | | |
| NM_005249.5_2965- | 128.74 | 185.93 | 27.65 | 153.10 |
| 2984_as_MOE_PO2_PO18 | | | | |
| NM_005249.5_2965- | 104.36 | 97.94 | 24.53 | 16.28 |
| 2984_as_MOE_PO3_PO17 | | | | |
| NM_005249.5_2965- | 116.93 | 123.02 | 48.97 | 22.67 |
| 2984_as_MOE_PO4_PO16 | | | | |
| NM_005249.5_2965- | 88.68 | 91.60 | 22.67 | 20.19 |
| 2984_as_MOE_PO5_PO15 | | | | |
| NM_005249.5_2965- | 101.24 | 100.16 | 25.12 | 10.94 |
| 2984_as_MOE_PO6_PO14 | | | | |
| NM_005249.5_2965- | 115.86 | 101.86 | 35.03 | 20.57 |
| 2984_as_MOE_PO7_PO13 | | | | |
| NM_005249.5_2965- | 106.60 | 104.60 | 32.16 | 11.06 |
| 2984_as_MOE_PO8_PO12 | | | | |
| NM_005249.5_2965- | 86.22 | 103.31 | 26.83 | 4.11 |
| 2984_as_MOE_PO9_PO11 | | | | |
| NM_005249.5_2965- | 75.57 | 130.64 | 21.46 | 40.51 |
| 2984_as_MOE_PO9_PO10 | | | | |
| NM_005249.5_2965- | 94.23 | 85.47 | 18.26 | 11.89 |
| 2984_as_MOE_PO10_PO11 | | | | |
| NM_005249.5_2965- | 125.32 | 111.98 | 40.72 | 35.60 |
| 2984_as_MOE_PO2_PO17 | | | | |
| NM_005249.5_2965- | 113.96 | 100.62 | 42.45 | 20.86 |
| 2984_as_MOE_PO3_PO18 | | | | |
| NM_005249.5_2965- | 79.64 | 144.59 | 8.40 | 10.09 |
| 2984_as_MOE_PO3_PO16 | | | | |
| NM_005249.5_2965- | 77.74 | 164.80 | 10.44 | 42.29 |
| 2984_as_MOE_PO4_PO17 | | | | |
| NM_005249.5_2965- | 77.97 | 103.96 | 5.85 | 25.42 |
| 2984_as_MOE_PO4_PO15 | | | | |
| NM_005249.5_2965- | 135.88 | 84.33 | 18.21 | 4.99 |
| 2984_as_MOE_PO5_PO16 | | | | |

Table 10 shows the result on FOXG1 expression in HEK293 cells associated with transfection with modified ASOs based on the NM_005249.5_2062-2081_as_MOE sequence. Transfection was performed with ASOs at concentrations of 50 nM and 5 nM. After 24h of incubation with ASOs, medium was removed, the cells were lysed, and QuantiGene detection was used to determine FOXG1 mRNA expression in cells lysates.

Individual expression values were scaled relative to the GAPDH housekeeping gene, then all normalized to control (mock transfected cell mean value and RLuc-transfected cells).

TABLE 10

| | % FOXG1 mRNA relative to GAPDH | | | |
|---|---|---|---|---|
| | Mean | | SD | |
| Name | 50 nM | 5 nM | 50 nM | 5 nM |
| NM_005249.5_2062- | 159.24 | 113.05 | 21.15 | 5.45 |
| 2081_as_MOE | | | | |
| NM_005249.5_2062- | 119.81 | 116.75 | 28.23 | 12.48 |
| 2081_as_MOE_L1 | | | | |
| NM_005249.5_2062- | 128.17 | 112.92 | 40.67 | 7.37 |
| 2081_as_MOE_L2 | | | | |
| NM_005249.5_2062- | 155.27 | 111.97 | 29.56 | 16.98 |
| 2081_as_MOE_L3 | | | | |
| NM_005249.5_2062- | 144.95 | 114.76 | 31.46 | 7.15 |
| 2081_as_MOE_L4 | | | | |
| NM_005249.5_2062- | 145.70 | 116.87 | 32.98 | 7.76 |
| 2081_as_MOE_L5 | | | | |
| NM_005249.5_2062- | 183.90 | 119.15 | 20.74 | 6.24 |
| 2081_as_MOE_L6 | | | | |
| NM_005249.5_2062- | 137.53 | 116.60 | 21.75 | 7.26 |
| 2081_as_MOE_L7 | | | | |
| NM_005249.5_2062- | 139.81 | 111.17 | 20.70 | 3.79 |
| 2081_as_MOE_L8 | | | | |
| NM_005249.5_2062- | 151.86 | 110.03 | 39.56 | 5.18 |
| 2081_as_MOE_L9 | | | | |
| NM_005249.5_2062- | 130.09 | 109.54 | 10.66 | 11.10 |
| 2081_as_MOE_L10 | | | | |
| NM_005249.5_2062- | 126.83 | 105.19 | 15.65 | 7.12 |
| 2081_as_MOE_L11 | | | | |
| NM_005249.5_2062- | 109.51 | 97.49 | 11.97 | 27.25 |
| 2081_as_MOE_L12 | | | | |
| NM_005249.5_2062- | 145.08 | 107.50 | 4.47 | 6.60 |
| 2081_as_MOE_L13 | | | | |
| NM_005249.5_2062- | 118.08 | 106.84 | 4.44 | 5.49 |
| 2081_as_MOE_L14 | | | | |
| NM_005249.5_2062- | 131.23 | 97.35 | 5.61 | 15.12 |
| 2081_as_MOE_L15 | | | | |
| NM_005249.5_2062- | 157.00 | 113.81 | 17.03 | 12.91 |
| 2081_as_MOE_L16 | | | | |
| NM_005249.5_2062- | 159.70 | 106.43 | 13.45 | 2.60 |
| 2081_as_MOE_L17 | | | | |
| NM_005249.5_2062- | 160.34 | 111.50 | 29.07 | 4.52 |
| 2081_as_MOE_L18 | | | | |
| NM_005249.5_2062- | 155.90 | 99.76 | 10.67 | 2.49 |
| 2081_as_MOE_L19 | | | | |
| NM_005249.5_2062- | 146.15 | 109.82 | 5.34 | 12.11 |
| 2081_as_MOE_L20 | | | | |
| NM_005249.5_2062- | 143.02 | 124.43 | 9.82 | 6.22 |
| 2081_as_MOE_L1_L20 | | | | |
| NM_005249.5_2062- | 153.41 | 110.01 | 7.23 | 11.88 |
| 2081_as_MOE_L2_L19 | | | | |
| NM_005249.5_2062- | 154.52 | 129.16 | 3.47 | 15.29 |
| 2081_as_MOE_L3_L18 | | | | |
| NM_005249.5_2062- | 152.70 | 115.12 | 10.42 | 29.16 |
| 2081_as_MOE_L4_L17 | | | | |
| NM_005249.5_2062- | 510.28 | 103.81 | 393.21 | 14.14 |
| 2081_as_MOE_L5_L16 | | | | |
| NM_005249.5_2062- | 143.76 | 94.85 | 8.67 | 9.44 |
| 2081_as_MOE_L6_L15 | | | | |
| NM_005249.5_2062- | 133.00 | 106.18 | 6.56 | 23.49 |
| 2081_as_MOE_L7_L14 | | | | |
| NM_005249.5_2062- | 130.48 | 90.45 | 35.61 | 18.24 |
| 2081_as_MOE_L8_L13 | | | | |
| NM_005249.5_2062- | 144.25 | 92.53 | 8.30 | 6.85 |
| 2081_as_MOE_L9_L12 | | | | |
| NM_005249.5_2062- | 134.56 | 107.58 | 3.12 | 18.97 |
| 2081_as_MOE_L10_L11 | | | | |
| NM_005249.5_2062- | 144.71 | 95.57 | 6.55 | 8.73 |
| 2081_as_MOE_PO2 | | | | |
| NM_005249.5_2062- | 142.99 | 93.09 | 10.51 | 14.60 |
| 2081_as_MOE_PO3 | | | | |
| NM_005249.5_2062- | 155.94 | 98.95 | 27.73 | 7.45 |
| 2081_as_MOE_PO4 | | | | |
| NM_005249.5_2062- | 150.01 | 91.15 | 4.62 | 15.31 |
| 2081_as_MOE_PO5 | | | | |
| NM_005249.5_2062- | 137.06 | 76.38 | 3.97 | 8.23 |
| 2081_as_MOE_PO6 | | | | |
| NM_005249.5_2062- | 156.20 | 83.41 | 9.28 | 8.42 |
| 2081_as_MOE_PO7 | | | | |
| NM_005249.5_2062- | 145.56 | 87.22 | 4.38 | 13.81 |
| 2081_as_MOE_PO8 | | | | |
| NM_005249.5_2062- | 158.01 | 83.74 | 5.96 | 22.50 |
| 2081_as_MOE_PO9 | | | | |
| NM_005249.5_2062- | 145.82 | 86.76 | 12.92 | 4.98 |
| 2081_as_MOE_PO10 | | | | |
| NM_005249.5_2062- | 138.22 | 80.61 | 20.16 | 17.09 |
| 2081_as_MOE_PO11 | | | | |
| NM_005249.5_2062- | 154.45 | 78.74 | 34.42 | 13.30 |
| 2081_as_MOE_PO12 | | | | |
| NM_005249.5_2062- | 147.59 | 80.87 | 19.87 | 12.47 |
| 2081_as_MOE_PO13 | | | | |
| NM_005249.5_2062- | 121.91 | 84.57 | 18.12 | 19.69 |
| 2081_as_MOE_PO14 | | | | |
| NM_005249.5_2062- | 143.12 | 76.22 | 19.01 | 11.99 |
| 2081_as_MOE_PO15 | | | | |
| NM_005249.5_2062- | 169.88 | 68.84 | 20.27 | 20.45 |
| 2081_as_MOE_PO16 | | | | |
| NM_005249.5_2062- | 167.44 | 74.00 | 7.89 | 19.85 |
| 2081_as_MOE_PO17 | | | | |
| NM_005249.5_2062- | 166.37 | 71.05 | 34.96 | 35.09 |
| 2081_as_MOE_PO18 | | | | |
| NM_005249.5_2062- | 158.91 | 76.14 | 27.55 | 19.02 |
| 2081_as_MOE_PO2_PO18 | | | | |
| NM_005249.5_2062- | 180.45 | 81.37 | 26.10 | 16.95 |
| 2081_as_MOE_PO3_PO17 | | | | |
| NM_005249.5_2062- | 154.05 | 94.45 | 18.23 | 13.33 |
| 2081_as_MOE_PO4_PO16 | | | | |
| NM_005249.5_2062- | 131.95 | 77.98 | 21.27 | 11.78 |
| 2081_as_MOE_PO5_PO15 | | | | |
| NM_005249.5_2062- | 133.57 | 79.93 | 9.61 | 3.86 |
| 2081_as_MOE_PO6_PO14 | | | | |
| NM_005249.5_2062- | 134.41 | 100.29 | 10.93 | 30.35 |
| 2081_as_MOE_PO7_PO13 | | | | |
| NM_005249.5_2062- | 138.80 | 74.29 | 26.24 | 6.33 |
| 2081_as_MOE_PO8_PO12 | | | | |
| NM_005249.5_2062- | 134.11 | 90.60 | 8.02 | 19.41 |
| 2081_as_MOE_PO9_PO11 | | | | |
| NM_005249.5_2062- | 128.20 | 81.20 | 3.46 | 3.86 |
| 2081_as_MOE_PO9_PO10 | | | | |
| NM_005249.5_2062- | 133.03 | 92.81 | 5.32 | 6.06 |
| 2081_as_MOE_PO10_PO11 | | | | |
| NM_005249.5_2062- | 164.23 | 85.12 | 44.37 | 11.68 |
| 2081_as_MOE_PO2_PO17 | | | | |
| NM_005249.5_2062- | 135.11 | 85.28 | 7.32 | 13.65 |
| 2081_as_MOE_PO3_PO18 | | | | |
| NM_005249.5_2062- | 272.94 | 91.46 | 176.00 | 4.12 |
| 2081_as_MOE_PO3_PO16 | | | | |
| NM_005249.5_2062- | 222.94 | 87.63 | 18.43 | 5.91 |
| 2081_as MOE PO4 PO17 | | | | |
| NM_005249.5_2062- | 130.27 | 83.19 | 17.11 | 1.09 |
| 2081_as_MOE_PO4_PO15 | | | | |
| NM_005249.5_2062- | 150.20 | 87.62 | 14.15 | 5.09 |
| 2081_as_MOE_PO5_PO16 | | | | |

Table 11 shows the result on FOXG1 expression in HEK293 cells associated with transfection with modified ASOs based on the NM_005249.5_2061-2080_as_MOE sequence. Transfection was performed with ASOs at concentrations of 50 nM and 5 nM. After 48h of incubation with ASOs, medium was removed, the cells were lysed, and QuantiGene detection was used to determine FOXG1 mRNA expression in cells lysates.

Individual expression values were scaled relative to the GAPDH housekeeping gene, then all normalized to control (mock transfected cell mean value and RLuc-transfected cells).

TABLE 11

| | % FOXG1 mRNA relative to GAPDH | | | |
|---|---|---|---|---|
| | Mean | | SD | |
| Name | 50 nM | 5 nM | 50 nM | 5 nM |
| NM_005249.5_2061- | 160.98 | 76.56 | 21.35 | 24.73 |
| 2080_as_MOE | | | | |
| NM_005249.5_2061- | 128.58 | 91.03 | 51.71 | 28.27 |
| 2080_as_MOE_L1 | | | | |
| NM_005249.5_2061- | 174.75 | 77.30 | 48.56 | 19.21 |
| 2080_as_MOE_L2 | | | | |
| NM_005249.5_2061- | 178.60 | 96.28 | 25.26 | 25.90 |
| 2080_as_MOE_L3 | | | | |
| NM_005249.5_2061- | 198.89 | 84.18 | 64.21 | 21.98 |
| 2080_as_MOE_L4 | | | | |
| NM_005249.5_2061- | 181.54 | 96.63 | 54.62 | 19.15 |
| 2080_as_MOE_L5 | | | | |
| NM_005249.5_2061- | 189.32 | 101.55 | 52.95 | 21.43 |
| 2080_as_MOE_L6 | | | | |
| NM_005249.5_2061- | 150.01 | 92.19 | 72.61 | 43.85 |
| 2080_as_MOE_L7 | | | | |
| NM_005249.5_2061- | 151.92 | 97.45 | 24.87 | 36.73 |
| 2080_as_MOE_L8 | | | | |
| NM_005249.5_2061- | 134.81 | 81.01 | 57.31 | 14.02 |
| 2080_as_MOE_L9 | | | | |
| NM_005249.5_2061- | 143.89 | 85.53 | 38.83 | 22.14 |
| 2080_as_MOE_L10 | | | | |
| NM_005249.5_2061- | 90.17 | 88.24 | 23.38 | 13.17 |
| 2080_as_MOE_L11 | | | | |
| NM_005249.5_2061- | 97.76 | 87.83 | 19.78 | 18.84 |
| 2080_as_MOE_L12 | | | | |
| NM_005249.5_2061- | 147.25 | 88.95 | 14.03 | 3.76 |
| 2080_as_MOE_L13 | | | | |
| NM_005249.5_2061- | 98.57 | 89.44 | 23.00 | 16.05 |
| 2080_as_MOE_L14 | | | | |
| NM_005249.5_2061- | 93.71 | 100.75 | 12.90 | 7.14 |
| 2080_as_MOE_L15 | | | | |
| NM_005249.5_2061- | 118.48 | 106.59 | 18.24 | 10.54 |
| 2080_as_MOE_L16 | | | | |
| NM_005249.5_2061- | 126.23 | 89.96 | 24.36 | 25.12 |
| 2080_as_MOE_L17 | | | | |
| NM_005249.5_2061- | 120.67 | 104.14 | 9.20 | 17.17 |
| 2080_as_MOE_L18 | | | | |
| NM_005249.5_2061- | 117.72 | 91.00 | 47.65 | 18.43 |
| 2080_as_MOE_L19 | | | | |
| NM_005249.5_2061- | 98.68 | 88.16 | 23.23 | 16.69 |
| 2080_as_MOE_L20 | | | | |
| NM_005249.5_2061- | 104.79 | 111.05 | 22.62 | 7.20 |
| 2080_as_MOE_L1_L20 | | | | |
| NM_005249.5_2061- | 191.78 | 92.00 | 41.18 | 2.36 |
| 2080_as_MOE_L2_L19 | | | | |
| NM_005249.5_2061- | 177.90 | 133.09 | 18.55 | 28.88 |
| 2080_as_MOE_L3_L18 | | | | |
| NM_005249.5_2061- | 143.67 | 125.62 | 15.36 | 22.39 |
| 2080_as_MOE_L4_L17 | | | | |
| NM_005249.5_2061- | 126.59 | 102.19 | 23.89 | 8.99 |
| 2080_as_MOE_L5_L16 | | | | |
| NM_005249.5_2061- | 158.52 | 114.55 | 9.45 | 16.57 |
| 2080_as_MOE_L6_L15 | | | | |
| NM_005249.5_2061- | 175.84 | 107.29 | 26.61 | 33.91 |
| 2080_as_MOE_L7_L14 | | | | |
| NM_005249.5_2061- | 135.05 | 91.92 | 27.76 | 25.62 |
| 2080_as_MOE_L8_L13 | | | | |
| NM_005249.5_2061- | 112.56 | 92.15 | 20.70 | 26.32 |
| 2080_as_MOE_L9_L12 | | | | |
| NM_005249.5_2061- | 129.40 | 96.95 | 40.92 | 23.91 |
| 2080_as_MOE_L10_L11 | | | | |
| NM_005249.5_2061- | 136.26 | 74.51 | 36.06 | 15.98 |
| 2080_as_MOE_PO2 | | | | |
| NM_005249.5_2061- | 106.48 | 76.44 | 28.23 | 8.72 |
| 2080_as_MOE_PO3 | | | | |
| NM_005249.5_2061- | 123.37 | 111.38 | 43.36 | 10.67 |
| 2080_as_MOE_PO4 | | | | |
| NM_005249.5_2061- | 120.49 | 100.18 | 49.89 | 19.50 |
| 2080_as_MOE_PO5 | | | | |
| NM_005249.5_2061- | 131.44 | 105.00 | 36.09 | 25.88 |
| 2080_as_MOE_PO6 | | | | |
| NM_005249.5_2061- | 158.36 | 87.75 | 32.98 | 34.16 |
| 2080_as_MOE_PO7 | | | | |
| NM_005249.5_2061- | 128.54 | 54.99 | 23.61 | 13.32 |
| 2080_as_MOE_PO8 | | | | |
| NM_005249.5_2061- | 143.94 | 88.76 | 23.37 | 17.56 |
| 2080_as_MOE_PO9 | | | | |
| NM_005249.5_2061- | 117.94 | 102.03 | 24.45 | 15.66 |
| 2080_as_MOE_PO10 | | | | |
| NM_005249.5_2061- | 161.39 | 95.03 | 21.15 | 17.49 |
| 2080_as_MOE_PO11 | | | | |
| NM_005249.5_2061- | 140.19 | 95.34 | 23.50 | 18.54 |
| 2080_as_MOE_PO12 | | | | |
| NM_005249.5_2061- | 135.10 | 89.96 | 14.09 | 20.93 |
| 2080_as_MOE_PO13 | | | | |
| NM_005249.5_2061- | 155.27 | 73.94 | 17.13 | 16.31 |
| 2080_as_MOE_PO14 | | | | |
| NM_005249.5_2061- | 136.56 | 85.21 | 36.56 | 11.95 |
| 2080_as_MOE_PO15 | | | | |
| NM_005249.5_2061- | 172.88 | 97.08 | 25.71 | 21.37 |
| 2080_as_MOE_PO16 | | | | |
| NM_005249.5_2061- | 186.00 | 97.59 | 23.80 | 5.43 |
| 2080_as_MOE_PO17 | | | | |
| NM_005249.5_2061- | 148.35 | 102.08 | 33.07 | 20.03 |
| 2080_as_MOE_PO18 | | | | |
| NM_005249.5_2061- | 179.06 | 100.72 | 29.12 | 13.98 |
| 2080_as_MOE_PO2_PO18 | | | | |
| NM_005249.5_2061- | 172.47 | 74.88 | 34.73 | 30.45 |
| 2080_as_MOE_PO3_PO17 | | | | |
| NM_005249.5_2061- | 120.83 | 85.35 | 17.18 | 7.59 |
| 2080_as_MOE_PO4_PO16 | | | | |
| NM_005249.5_2061- | 132.37 | 83.68 | 22.89 | 12.93 |
| 2080_as_MOE_PO5_PO15 | | | | |
| NM_005249.5_2061- | 146.31 | 85.50 | 12.99 | 23.17 |
| 2080_as_MOE_PO6_PO14 | | | | |
| NM_005249.5_2061- | 131.72 | 89.22 | 15.44 | 20.04 |
| 2080_as_MOE_PO7_PO13 | | | | |
| NM_005249.5_2061- | 150.16 | 80.38 | 18.56 | 6.70 |
| 2080_as_MOE_PO8_PO12 | | | | |
| NM_005249.5_2061- | 139.22 | 68.35 | 11.78 | 27.04 |
| 2080_as_MOE_PO9_PO11 | | | | |
| NM_005249.5_2061- | 169.77 | 98.11 | 39.80 | 21.56 |
| 2080_as_MOE_PO9_PO10 | | | | |
| NM_005249.5_2061- | 155.39 | 109.75 | 12.69 | 20.62 |
| 2080_as_MOE_PO10_PO11 | | | | |
| NM_005249.5_2061- | 188.49 | 83.55 | 20.20 | 12.63 |
| 2080_as_MOE_PO2_PO17 | | | | |
| NM_005249.5_2061- | 193.67 | 91.05 | 77.50 | 9.76 |
| 2080_as_MOE_PO3_PO18 | | | | |
| NM_005249.5_2061- | 156.96 | 74.57 | 34.47 | 6.02 |
| 2080_as_MOE_PO3_PO16 | | | | |
| NM_005249.5_2061- | 148.62 | 70.53 | 56.19 | 19.49 |
| 2080_as_MOE_PO4_PO17 | | | | |
| NM_005249.5_2061- | 141.57 | 65.09 | 20.29 | 14.52 |
| 2080_as_MOE_PO4_PO15 | | | | |
| NM_005249.5_2061- | 134.82 | 86.35 | 16.94 | 11.53 |
| 2080_as_MOE_PO5_PO16 | | | | |

Table 12 shows data for select modified ASO associated with the modulation of FOXG1 expression in CFF-STTG1 cell lines.

TABLE 12
ASO targets resulting in upregulation
of FOXG1 mRNA in CFF-STTG1 cells

| | % FOXG1 mRNA relative to GAPDH | | | |
|---|---|---|---|---|
| | Mean | | SD | |
| Name | 50 nM | 5 nM | 50 nM | 5 nM |

NM_005249.5_2062-2081_as variants

| | | | | |
|---|---|---|---|---|
| NM_005249.5_2062- | 144.20 | 131.27 | 36.10 | 31.45 |
| 2081_as_MOE | | | | |
| NM_005249.5_2062- | 141.11 | 125.11 | 16.72 | 25.40 |
| 2081_as_MOE_L3 | | | | |
| NM_005249.5_2062- | 149.70 | 124.66 | 12.96 | 12.30 |
| 2081_as_MOE_L6 | | | | |
| NM_005249.5_2062- | 149.76 | 135.73 | 27.86 | 28.16 |
| 2081_as_MOE_L2_L19 | | | | |
| NM_005249.5_2062- | 126.62 | 104.72 | 9.19 | 11.64 |
| 2081_as_MOE_L3_L18 | | | | |
| NM_005249.5_2062- | 131.58 | 134.68 | 11.02 | 14.38 |
| 2081_as_MOE_L4_L17 | | | | |
| NM_005249.5_2062- | 147.41 | 142.21 | 10.91 | 17.30 |
| 2081_as_MOE_L5_L16 | | | | |
| NM_005249.5_2062- | 132.37 | 131.04 | 10.53 | 14.73 |
| 2081_as_MOE_PO3_PO16 | | | | |
| NM_005249.5_2062- | 134.04 | 124.00 | 15.45 | 18.03 |
| 2081_as_MOE_PO4_PO17 | | | | |

NM_005249.5_2965-2984_as variants

| | | | | |
|---|---|---|---|---|
| NM_005249.5_2965- | 75.95 | 126.89 | 10.85 | 14.72 |
| 2984_as_MOE | | | | |
| NM_005249.5_2965- | 110.52 | 128.06 | 27.62 | 24.75 |
| 2984_as_MOE_L3 | | | | |
| NM_005249.5_2965- | 107.07 | 141.12 | 29.76 | 25.44 |
| 2984_as_MOE_L4 | | | | |
| NM_005249.5_2965- | 87.60 | 133.13 | 30.31 | 28.24 |
| 2984_as_MOE_L8 | | | | |
| NM_005249.5_2965- | 88.58 | 141.25 | 18.02 | 21.78 |
| 2984_as_MOE_PO2_PO18 | | | | |

NM_005249.5_2061-2080_as Variants

| | | | | |
|---|---|---|---|---|
| NM_005249.5_2061- | 144.20 | 131.27 | 36.10 | 31.45 |
| 2080_as_MOE | | | | |
| NM_005249.5_2061- | 160.02 | 119.87 | 20.52 | 28.35 |
| 2080_as_MOE_L6 | | | | |
| NM_005249.5_2061- | 153.57 | 114.43 | 16.67 | 18.84 |
| 2080_as_MOE_L2_L19 | | | | |
| NM_005249.5_2061- | 171.07 | 116.95 | 15.72 | 12.93 |
| 2080_as_MOE_L3_L18 | | | | |
| NM_005249.5_2061- | 153.79 | 112.17 | 24.34 | 30.94 |
| 2080_as_MOE_L6_L15 | | | | |
| NM_005249.5_2061- | 156.40 | 127.36 | 27.82 | 33.71 |
| 2080_as_MOE_L7_L14 | | | | |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

SEQUENCES

| SEQ ID NO | SEQUENCE |
|---|---|
| 1 | AGCGATCGAGGCGGCTATAG |
| 2 | CAGCGATCGAGGCGGCTATA |
| 3 | ACAGCGATCGAGGCGGCTAT |
| 4 | GACAGCGATCGAGGCGGCTA |
| 5 | AGACAGCGATCGAGGCGGCT |
| 6 | GCAGCAGTCACAGCAGCAGC |
| 7 | CGCAGCAGCAGTCACAGCAG |
| 8 | TCGCAGCAGCAGTCACAGCA |
| 9 | CTCGCAGCAGCAGTCACAGC |
| 10 | TCTCGCAGCAGCAGTCACAG |
| 11 | CTCTCGCAGCAGCAGTCACA |
| 12 | CCTCTCGCAGCAGCAGTCAC |
| 13 | TCCTCTCGCAGCAGCAGTCA |
| 14 | CTCCTCTCGCAGCAGCAGTC |
| 15 | CCTCCTCTCGCAGCAGCAGT |
| 16 | TCCTCCTCTCGCAGCAGCAG |
| 17 | CTCCTCCTCTCGCAGCAGCA |
| 18 | TCCTCCTCCTCTCGCAGCAG |
| 19 | CTCCTCCTCCTCTCGCAGCA |
| 20 | TCCTCCTCCTCCTCTCGCAG |
| 21 | CTCCTCCTCCTCCTCTCGCA |
| 22 | GCTGCTTCCTCCTCCTCCTC |
| 23 | CGCTGCTTCCTCCTCCTCCT |
| 24 | TGTACTTCTTGGTCTCCCCC |
| 25 | CTGTACTTCTTGGTCTCCCC |
| 26 | ACTGTACTTCTTGGTCTCCC |
| 27 | AACTGTACTTCTTGGTCTCC |
| 28 | CAACTGTACTTCTTGGTCTC |
| 29 | CCAACTGTACTTCTTGGTCT |
| 30 | CCCAACTGTACTTCTTGGTC |
| 31 | TCCCAACTGTACTTCTTGGT |
| 32 | CTCCCAACTGTACTTCTTGG |
| 33 | GCTCCCAACTGTACTTCTTG |
| 34 | CGCTCCCAACTGTACTTCTT |
| 35 | TCGCTCCCAACTGTACTTCT |
| 36 | CTCGCTCCCAACTGTACTTC |
| 37 | CCTCGCTCCCAACTGTACTT |
| 38 | CCCTCGCTCCCAACTGTACT |
| 39 | TCCCTCGCTCCCAACTGTAC |
| 40 | CTCCCTCGCTCCCAACTGTA |
| 41 | GCTCCCTCGCTCCCAACTGT |
| 42 | AGCTCCCTCGCTCCCAACTG |
| 43 | AAGCTCCCTCGCTCCCAACT |
| 44 | GAAGCTCCCTCGCTCCCAAC |
| 45 | TGAAGCTCCCTCGCTCCCAA |
| 46 | GTGAAGCTCCCTCGCTCCCA |
| 47 | AAGAAACAACCACCGCCCCG |
| 48 | AAAGAAACAACCACCGCCCC |
| 49 | AAAAGAAACAACCACCGCCC |
| 50 | AAAAAGAAACAACCACCGCC |
| 51 | CCCCTCAGGAATTAGAAAAA |
| 52 | ACCCCTCAGGAATTAGAAAA |
| 53 | CACCCCTCAGGAATTAGAAA |
| 54 | CCACCCCTCAGGAATTAGAA |
| 55 | ACCACCCCTCAGGAATTAGA |
| 56 | AACCACCCCTCAGGAATTAG |
| 57 | CAACCACCCCTCAGGAATTA |
| 58 | GCAACCACCCCTCAGGAATT |
| 59 | AGCAACCACCCCTCAGGAAT |
| 60 | CAGCAACCACCCCTCAGGAA |
| 61 | GCAGCAACCACCCCTCAGGA |
| 62 | AAGCAGCAACCACCCCTCAG |
| 63 | AAAGCAGCAACCACCCCTCA |
| 64 | AAAAGCAGCAACCACCCCTC |
| 65 | CAAAAGCAGCAACCACCCCT |
| 66 | GCAAAAGCAGCAACCACCCC |
| 67 | AGCAAAAGCAGCAACCACCC |
| 68 | TAGCAAAAGCAGCAACCACC |
| 69 | GTAGCAAAAGCAGCAACCAC |
| 70 | TGTAGCAAAAGCAGCAACCA |
| 71 | ATGTAGCAAAAGCAGCAACC |
| 72 | CATGTAGCAAAAGCAGCAAC |
| 73 | TCATGTAGCAAAAGCAGCAA |
| 74 | GTCATGTAGCAAAAGCAGCA |
| 75 | AGTCATGTAGCAAAAGCAGC |
| 76 | AAGTCATGTAGCAAAAGCAG |
| 77 | CAAGTCATGTAGCAAAAGCA |
| 78 | GCAAGTCATGTAGCAAAAGC |
| 79 | GGCAAGTCATGTAGCAAAAG |
| 80 | TGGCAAGTCATGTAGCAAAA |
| 81 | CTGGCAAGTCATGTAGCAAA |
| 82 | GCTGGCAAGTCATGTAGCAA |
| 83 | CGCTGGCAAGTCATGTAGCA |
| 84 | GCGCTGGCAAGTCATGTAGC |
| 85 | TCACTTACAGTCTGGTCCCA |
| 86 | TTCACTTACAGTCTGGTCCC |
| 87 | ACGTTCACTTACAGTCTGGT |
| 88 | GTGTAAAACGTTCACTTACA |
| 89 | TGTGTAAAACGTTCACTTAC |
| 90 | GTGTGTAAAACGTTCACTTA |
| 91 | TGTGTGTAAAACGTTCACTT |
| 92 | TGCAAATGTGTGTAAAACGT |
| 93 | ATGCAAATGTGTGTAAAACG |
| 94 | AATGCAAATGTGTGTAAAAC |
| 95 | CAATGCAAATGTGTGTAAAA |
| 96 | TTTACAATGCAAATGTGTGT |
| 97 | AAATACCTGGACTTATTTTT |
| 98 | AAAATACCTGGACTTATTTT |
| 99 | AAAAATACCTGGACTTATTT |
| 100 | AACGTACAGAAATGGGAGGG |
| 101 | AAACGTACAGAAATGGGAGG |
| 102 | CAAACGTACAGAAATGGGAG |
| 103 | ACAAACGTACAGAAATGGGA |
| 104 | AACAAACGTACAGAAATGGG |
| 105 | GAACAAACGTACAGAAATGG |
| 106 | CACTCCACACCTTGTTAGAA |
| 107 | ACACTCCACACCTTGTTAGA |
| 108 | GACACTCCACACCTTGTTAG |
| 109 | TCGCTGACACTCCACACCTT |
| 110 | GTATTCTCCCCACATTGCAC |
| 111 | TGTATTCTCCCCACATTGCA |
| 112 | ATGTATTCTCCCCACATTGC |
| 113 | ACAATGTATTCTCCCCACAT |
| 114 | TTGACTTCCAAACCTTATAT |
| 115 | TTTGACTTCCAAACCTTATA |
| 116 | CTACTATAATTTGACTTCCA |
| 117 | TCTACTATAATTTGACTTCC |
| 118 | TTCTACTATAATTTGACTTC |
| 119 | CATTCTACTATAATTTGACT |
| 120 | ACATTCTACTATAATTTGAC |
| 121 | GATACACATTCTACTATAAT |
| 122 | AGATACACATTCTACTATAA |
| 123 | TAGATACACATTCTACTATA |
| 124 | TTAGATACACATTCTACTAT |
| 125 | TTTAGATACACATTCTACTA |
| 126 | ATTTAGATACACATTCTACT |
| 127 | TATTTAGATACACATTCTAC |
| 128 | CTATTTAGATACACATTCTA |
| 129 | CACTATTTAGATACACATTC |
| 130 | GTCACTATTTAGATACACAT |
| 131 | AGTCACTATTTAGATACACA |
| 132 | CAGTCACTATTTAGATACAC |
| 133 | AGCAGTCACTATTTAGATAC |
| 134 | AAGCAGTCACTATTTAGATA |
| 135 | AAAGCAGTCACTATTTAGAT |
| 136 | CAAAGCAGTCACTATTTAGA |
| 137 | GCAAAGCAGTCACTATTTAG |
| 138 | GGCAAAGCAGTCACTATTTA |
| 139 | TGGCAAAGCAGTCACTATTT |
| 140 | AATGGCAAAGCAGTCACTAT |
| 141 | AAATGGCAAAGCAGTCACTA |
| 142 | GAAATGGCAAAGCAGTCACT |
| 143 | AATGAAATGGCAAAGCAGTC |
| 144 | AGGTTTGAATGAAATGGCAA |
| 145 | CAGGTTTGAATGAAATGGCA |
| 146 | TCAGGTTTGAATGAAATGGC |
| 147 | GTCAGGTTTGAATGAAATGG |
| 148 | CTTGTCAGGTTTGAATGAAA |
| 149 | CTTAGAGATAGACTTGTCAG |
| 150 | TCTTAGAGATAGACTTGTCA |
| 151 | CTCTTAGAGATAGACTTGTC |
| 152 | GCTCTTAGAGATAGACTTGT |
| 153 | GGCTCTTAGAGATAGACTTG |
| 154 | CGGCTCTTAGAGATAGACTT |
| 155 | GCGGCTCTTAGAGATAGACT |
| 156 | TGGCGGCTCTTAGAGATAGA |
| 157 | TCTGGCGGCTCTTAGAGATA |
| 158 | ATCTGGCGGCTCTTAGAGAT |
| 159 | AATCTGGCGGCTCTTAGAGA |
| 160 | TACTGCACACATGGAAATCT |
| 161 | ATACTGCACACATGGAAATC |
| 162 | AATACTGCACACATGGAAAT |
| 163 | ATAATACTGCACACATGGAA |
| 164 | CTTATAATACTGCACACATG |
| 165 | AACTTATAATACTGCACACA |
| 166 | TAACTTATAATACTGCACAC |
| 167 | ATAACTTATAATACTGCACA |
| 168 | GATAACTTATAATACTGCAC |
| 169 | TGATAACTTATAATACTGCA |
| 170 | ATGATAACTTATAATACTGC |
| 171 | GTTCCATGATAACTTATAAT |
| 172 | AGTTCCATGATAACTTATAA |
| 173 | TAGTTCCATGATAACTTATA |
| 174 | ATAGTTCCATGATAACTTAT |
| 175 | TATAGTTCCATGATAACTTA |
| 176 | TCTGCGTCCACCATATAGTT |
| 175 | GTCTGCGTCCACCATATAGT |
| 178 | GGTCTGCGTCCACCATATAG |
| 179 | AGGTCTGCGTCCACCATATA |
| 180 | AAGGTCTGCGTCCACCATAT |
| 181 | TTCTCAAGGTCTGCGTCCAC |
| 182 | GTTCTCAAGGTCTGCGTCCA |
| 183 | TGTTCTCAAGGTCTGCGTCC |
| 184 | TTGTTCTCAAGGTCTGCGTC |
| 185 | GTTGTTCTCAAGGTCTGCGT |
| 186 | GGTTGTTCTCAAGGTCTGCG |
| 187 | AGGTTGTTCTCAAGGTCTGC |
| 188 | TAGGTTGTTCTCAAGGTCTG |
| 189 | TTAGGTTGTTCTCAAGGTCT |
| 190 | TTTAGGTTGTTCTCAAGGTC |
| 191 | AATTTAGGTTGTTCTCAAGG |
| 192 | CCCATAATTTAGGTTGTTCT |
| 193 | CCCCATAATTTAGGTTGTTC |
| 194 | TCCCCATAATTTAGGTTGTT |
| 195 | CTCCCCATAATTTAGGTTGT |
| 196 | TCTCCCCATAATTTAGGTTG |
| 197 | AAATTCTCCCCATAATTTAG |
| 198 | CAATAAATGGCCAAAATAAT |
| 199 | TCTTTGGTCTAAAAGTAAAC |
| 200 | ATCTTTGGTCTAAAAGTAAA |
| 201 | AATCTTTGGTCTAAAAGTAA |
| 202 | CAATCTTTGGTCTAAAAGTA |
| 203 | TTTCTAGAACCCAATCTTTG |
| 204 | CATTTTCTAGAACCCAATCT |
| 205 | GCATTTTCTAGAACCCAATC |
| 206 | TGCATTTTCTAGAACCCAAT |
| 207 | GTGCATTTTCTAGAACCCAA |
| 208 | AGTGCATTTTCTAGAACCCA |
| 209 | CAAGTGCATTTTCTAGAACC |
| 210 | CCAAGTGCATTTTCTAGAAC |
| 211 | ACCAAGTGCATTTTCTAGAA |
| 212 | TACCAAGTGCATTTTCTAGA |
| 213 | ATACCAAGTGCATTTTCTAG |
| 214 | TATACCAAGTGCATTTTCTA |
| 215 | GTATACCAAGTGCATTTTCT |
| 216 | AGTATACCAAGTGCATTTTC |
| 217 | TAGTATACCAAGTGCATTTT |
| 218 | TTAGTATACCAAGTGCATTT |
| 219 | ACTTAGTATACCAAGTGCAT |
| 220 | TACTTAGTATACCAAGTGCA |
| 221 | ATACTTAGTATACCAAGTGC |
| 222 | AATACTTAGTATACCAAGTG |
| 223 | GTTTTAATACTTAGTATACC |
| 224 | AGTGTTGCCAACTGAAACAA |
| 225 | CAATTGAATGGGCAGTGTTG |
| 226 | TCAATTGAATGGGCAGTGTT |
| 227 | TTCAATTGAATGGGCAGTGT |
| 228 | TGAAGGCAATCGTTAATTTT |
| 229 | CTGAAGGCAATCGTTAATTT |
| 230 | ACTGAAGGCAATCGTTAATT |
| 231 | AACTGAAGGCAATCGTTAAT |
| 232 | AAACTGAAGGCAATCGTTAA |
| 233 | CAAACTGAAGGCAATCGTTA |
| 234 | ACAAACTGAAGGCAATCGTT |
| 235 | ACACAAACTGAAGGCAATCG |
| 236 | GTGACCACATACATCAAAAT |
| 237 | TTAGTGACCACATACATCAA |
| 238 | TTTACCTATAAGTACAATAG |
| 239 | GTTTACCTATAAGTACAATA |
| 240 | GGTTTACCTATAAGTACAAT |
| 241 | ACATATTTGCAAGGTTTACC |
| 242 | TACATATTTGCAAGGTTTAC |
| 243 | TTACATATTTGCAAGGTTTA |
| 244 | GTTACATATTTGCAAGGTTT |
| 245 | GGTTACATATTTGCAAGGTT |
| 246 | AGGTTACATATTTGCAAGGT |
| 247 | CAGGTTACATATTTGCAAGG |
| 248 | ACAGGTTACATATTTGCAAG |
| 249 | ACACAGGTTACATATTTGCA |
| 250 | AACACAGGTTACATATTTGC |
| 251 | GCAACACAGGTTACATATTT |
| 252 | GCGCAACACAGGTTACATAT |
| 253 | TGCGCAACACAGGTTACATA |
| 254 | TTGCGCAACACAGGTTACAT |
| 255 | TTTGCGCAACACAGGTTACA |
| 256 | CATTTGCGCAACACAGGTTA |
| 257 | ACTCAAATTTATGCGGCATT |
| 258 | ATCACTCAAATTTATGCGGC |
| 259 | ACATTAACAATCACTCAAAT |
| 260 | CAACATTAACAATCACTCAA |
| 261 | ACAACATTAACAATCACTCA |
| 262 | GACAACATTAACAATCACTC |
| 263 | AGACAACATTAACAATCACT |
| 264 | ACCACAGTATCACAATCAAG |
| 265 | GACCACAGTATCACAATCAA |
| 266 | TGACCACAGTATCACAATCA |
| 267 | ATGACCACAGTATCACAATC |
| 268 | CATATGACCACAGTATCACA |
| 269 | GCATATGACCACAGTATCAC |
| 270 | GACAAACACGGGCATATGAC |
| 271 | TGACAAACACGGGCATATGA |
| 272 | GTTCATAGTAAACATTTTTG |
| 273 | GTGTTCATAGTAAACATTTT |
| 274 | TGTGTTCATAGTAAACATTT |
| 275 | TCTGTGTGTTCATAGTAAAC |
| 276 | TTCTGTGTGTTCATAGTAAA |
| 277 | TATTTCTGTGTGTTCATAGT |
| 278 | GATATATATGAATTTAGCCT |
| 279 | AGATATATATGAATTTAGCC |
| 280 | AGACAAAAGTATCAAGATAT |
| 281 | AGTTGATTGGTCTTTAAAAA |
| 282 | CCCTATAAGTTGATTGGTCT |
| 283 | AAAAAGCCTTTGAATTCCCT |
| 284 | TAAATTTTAGTTTGGCTGAA |
| 285 | TTAAATTTTAGTTTGGCTGA |
| 286 | TTTAAATTTTAGTTTGGCTG |
| 287 | GTTTAAATTTTAGTTTGGCT |
| 288 | TTAGAGTCAGTTCAAATTAA |
| 289 | TTTAGAGTCAGTTCAAATTA |
| 290 | TTTTAGAGTCAGTTCAAATT |
| 291 | TCATTTTTAGAGTCAGTTCA |
| 292 | TTCATTTTTAGAGTCAGTTC |
| 293 | GTTCACAAAGGGAAAAATAC |
| 294 | CTGCTCCTTGTAAAATTTGT |
| 295 | GCTGCTCCTTGTAAAATTTG |
| 296 | TGTTTATTAAATAGGCTGCT |
| 297 | GTGTTTATTAAATAGGCTGC |
| 298 | TAGTGTTTATTAAATAGGCT |
| 299 | CTAGTGTTTATTAAATAGGC |
| 300 | GCTAGTGTTTATTAAATAGG |
| 301 | AAAGCCTATACTTTGTTTAA |
| 302 | TCAGCTGAAAAGCCTATACT |
| 303 | ATCAGCTGAAAAGCCTATAC |
| 304 | TATCAGCTGAAAAGCCTATA |
| 305 | GTATCAGCTGAAAAGCCTAT |
| 306 | GGTATCAGCTGAAAAGCCTA |
| 307 | TGTATATCCACAGAAACTTA |
| 308 | CTTTTTGCTGTATATCCACA |
| 309 | TCTTTTTGCTGTATATCCAC |
| 310 | CTCTTTTTGCTGTATATCCA |
| 311 | TCTCTTTTTGCTGTATATCC |
| 312 | ATCTCTTTTTGCTGTATATC |
| 313 | ATATCTCTTTTTGCTGTATA |
| 314 | TATATCTCTTTTTGCTGTAT |
| 315 | TTATATCTCTTTTTGCTGTA |
| 316 | ATTATATCTCTTTTTGCTGT |
| 317 | AATTATATCTCTTTTTGCTG |
| 318 | GGTAAAGAGCTATGCACAGA |
| 319 | GGGTAAAGAGCTATGCACAG |
| 320 | AGGGTAAAGAGCTATGCACA |
| 321 | CAGGGTAAAGAGCTATGCAC |
| 322 | ACAGGGTAAAGAGCTATGCA |
| 323 | AACACAGGGTAAAGAGCTAT |
| 324 | GCCAAGCTCTATTAACAATA |
| 325 | TGCCAAGCTCTATTAACAAT |
| 326 | TTGCCAAGCTCTATTAACAA |
| 327 | TTTGCCAAGCTCTATTAACA |
| 328 | ATAATTTGCCAAGCTCTATT |
| 329 | TATAATTTGCCAAGCTCTAT |
| 330 | TTATAATTTGCCAAGCTCTA |
| 331 | ATTTATAATTTGCCAAGCTC |
| 332 | TATTTATAATTTGCCAAGCT |
| 333 | TTATTTATAATTTGCCAAGC |
| 334 | ACTTCTATCTAACCATATAC |
| 335 | GTCACTTCTATCTAACCATA |
| 336 | AGTCACTTCTATCTAACCAT |
| 337 | TAGTCACTTCTATCTAACCA |
| 338 | ATAGTCACTTCTATCTAACC |
| 339 | TATAGTCACTTCTATCTAAC |
| 340 | TTATAGTCACTTCTATCTAA |
| 341 | ATTATAGTCACTTCTATCTA |
| 342 | CATTATAGTCACTTCTATCT |
| 343 | GCATTATAGTCACTTCTATC |
| 344 | TGCATTATAGTCACTTCTAT |
| 345 | GTGCATTATAGTCACTTCTA |
| 346 | GGGCTCTGTGTGTCTATATA |
| 347 | AGGGCTCTGTGTGTCTATAT |
| 348 | AAGGGCTCTGTGTGTCTATA |
| 349 | GAAGGGCTCTGTGTGTCTAT |
| 350 | TGAAGGGCTCTGTGTGTCTA |
| 351 | ACTGAAGGGCTCTGTGTGTC |
| 352 | GAACTGAAGGGCTCTGTGTG |
| 353 | TGAACTGAAGGGCTCTGTGT |
| 354 | CTGAACTGAAGGGCTCTGTG |
| 355 | CCTGAACTGAAGGGCTCTGT |
| 356 | AAATTGTACCTGAACTGAAG |
| 357 | CAAATTGTACCTGAACTGAA |
| 358 | GCAAATTGTACCTGAACTGA |
| 359 | CGCAAATTGTACCTGAACTG |
| 360 | GCGCAAATTGTACCTGAACT |
| 361 | ATAAATGCTGACTTAGAAAG |
| 362 | AAATAAATGCTGACTTAGAA |
| 363 | AAAATAAATGCTGACTTAGA |
| 364 | GTGGGTAAACAGCCACAAAA |
| 365 | TGTGGGTAAACAGCCACAAA |
| 366 | ATTGTGGGTAAACAGCCACA |
| 367 | CATTGTGGGTAAACAGCCAC |
| 368 | TCATTGTGGGTAAACAGCCA |
| 369 | TTCATTGTGGGTAAACAGCC |
| 370 | TTTCATTGTGGGTAAACAGC |
| 371 | CTTTCATTGTGGGTAAACAG |
| 372 | TCTTTCATTGTGGGTAAACA |
| 373 | CTCTTTCATTGTGGGTAAAC |
| 374 | ACTCTTTCATTGTGGGTAAA |
| 375 | AACTCTTTCATTGTGGGTAA |
| 376 | GAACTCTTTCATTGTGGGTA |
| 377 | AGAACTCTTTCATTGTGGGT |
| 378 | TAGAACTCTTTCATTGTGGG |
| 379 | TTAGAACTCTTTCATTGTGG |
| 380 | CTTTATTAGAACTCTTTCAT |
| 381 | ACATCTTTATTAGAACTCTT |
| 382 | GCACATCTTTATTAGAACTC |
| 383 | CAGCACATCTTTATTAGAAC |
| 384 | TCAGCACATCTTTATTAGAA |

1. A modified antisense oligonucleotide (ASO), comprising a sequence complementary to a target nucleic acid sequence of a FOXG1 nucleic acid.

2. The modified ASO of claim 1, wherein the modified ASO comprises one or more modifications to any one of SEQ ID NO. 100, SEQ ID NO 101, or SEQ ID NO. 284.

3. The modified ASO of claim 1 or claim 2, wherein ASO comprises a modification within an inter-nucleoside linker or within a nucleoside.

4. The modified ASO of claim 3, wherein the modification comprises a modified inter-nucleoside linker and a modified nucleoside.

5. The modified ASO of any one of claims 1-4, wherein the modified ASO comprises at least 1 to 10 modified inter-nucleoside linkers.

6. The modified ASO of any one of claims 1-4, wherein the modified ASO comprises at least 10 to 20 modified inter-nucleoside linkers.

7. The modified antisense oligonucleotide of any one of claims 1-5, wherein the modified ASO comprises at least 1 to 10 modified nucleosides.

8. The modified ASO of any one of claims 1-5, wherein the modified ASO comprises at least 10 to 20 modified nucleosides.

9. The modified ASO of any one of claims 1-8, wherein the modified ASO comprises at least 10%, at least 25%, at least 50%, at least 75%, at least 80%, or at least 90% modified inter-nucleoside linkers.

10. The modified ASO of any one of claims 1-9, wherein the modified ASO comprises at least 10%, at least 25%, at least 50%, at least 75%, at least 80%, or at least 90% modified nucleosides.

11. The modified ASO of any one of claims 1-9, wherein the modified ASO comprises 16 nucleotides, 17 nucleotides, 18 nucleotides, 19 nucleotides, 20 nucleotides, 21 nucleotides, 22 nucleotides, or 23 nucleotides.

12. The modified ASO of any one of claims 1 to 11, wherein the FOXG1 nucleic acid comprises a 5′ untranslated region (5′ UTR) and a 3′ untranslated region (3′ UTR), and wherein the target sequence is located at the 5′ UTR or the 3′ UTR of the FOXG1 nucleic acid.

13. The modified ASO of claim 12, wherein the modified ASO hybridizes to a region of FOXG1 selected from any one of the regions of Table 1 or Table 2.

14. The modified ASO of claim 13, wherein the target sequence is located within a NM_005249.5_2000-2200_as region of the FOXG1 nucleic acid.

15. The modified ASO of claim 13, wherein the target sequence is located within a NM_005249.5_2900-3000_as region of the FOXG1 nucleic acid.

16. The modified ASO of claim 13, wherein the target sequence is NM_005249.5_2965-2984, NM_005249.5_2062-2081, or NM_005249.5_2061-2080.

17. The modified ASO of any one of claims 1 to 16, wherein the modified ASO is a single-stranded modified oligonucleotide.

18. The modified ASO of any one of claims 1 to 16, wherein the FOXG1 nucleic acid molecule is a ribonucleic acid (RNA).

19. The modified ASO of claim 18, wherein the RNA molecule is a messenger RNA (mRNA) molecule.

20. The modified ASO of any one of claims 1 to 19, wherein the modified ASO inhibits regulatory elements that reduce translation of the FOXG1 RNA.

21. The modified ASO of any one of claims 1 to 19, wherein the modified ASO inhibits regulatory elements that reduce stability of the FOXG1 RNA.

22. The modified ASO of claim 19, wherein the modified ASO inhibits regulatory elements located within the 3′ UTR of the FOXG1 RNA.

23. The modified ASO of claim 19, wherein the modified ASO sterically inhibits (1) miRNA binding and suppression of FOXG1 translation and/or (2) an RNA binding protein from binding to a regulatory sequence of the FOXG1 RNA and destabilizing the FOXG1 RNA.

24. The modified ASO of claim 19, wherein the modified ASO inhibits nuclease digestion of the FOXG1 RNA.

25. The modified ASO of any one of claims 1 to 24, wherein the modified ASO comprises one or more modifications selected from the group consisting of: a phosphorothioate linkage; a 2′-O-methoxy-ethyl A; a 2′-O-methoxy-ethyl T; a 2′-O-methoxy-ethyl C; a 2′-O-methoxy-ethyl G, a locked nucleic acid (LNA) A; a LNA T; a LNA C; and a LNA G.

26. The modified ASO of any one of claims 1 to 25, wherein the modified ASO comprises one of the sequences set forth in Table 6, Table 7, Table 8, or Table 12.

27. A pharmaceutical composition comprising the modified ASO of any one of claims 1 to 26 and a pharmaceutically acceptable carrier or diluent.

28. A method of modulating expression of a FOXG1 in a cell, comprising contacting the cell with the modified ASO of any one of claims 1 to 26.

29. The method of claim 28, wherein the cell is a located in a brain of an individual.

30. The method of claim 29, wherein the individual is a human.

31. The method of claim 29, wherein the individual comprises a mutated FOXG1 gene.

32. The method of claim 29, wherein the individual has a FOXG1 disease or disorder.

33. The method of claim 32, wherein the FOXG1 disease or disorder is FOXG1 syndrome.

34. The method of any one of claims 29 to 33, wherein the FOXG1 nucleic acid is a ribonucleic acid (RNA).

35. The method of claim 34, wherein the RNA is a messenger RNA (mRNA).

36. The method of any one of claims 29 to 35, wherein the modified ASO inhibits regulatory elements that reduce translation or stability of the FOXG1 RNA, thereby increasing an amount of FOXG1 protein in the cell.

37. The method of any one of claims 29 to 36, wherein modulating expression comprises increasing expression of a FOXG1 protein in the cell.

38. The method of any one of claims 29 to 36, wherein modulating expression comprises increasing stability or half-life of the FOXG1 nucleic acid in the cell.

39. The method of any one of claims 29 to 36, wherein modulating expression comprises increasing translation of a FOXG1 protein in the cell.

40. The method of any one of claims 29 to 39, wherein the modified ASO is administered to the individual by intrathecal injection, intracerebroventricular injection, inhalation, parenteral injection or infusion, or orally.

41. A method of treating or ameliorating a FOXG1 disease or disorder in an individual having, or at risk of having, the FOXG1 disease or disorder, comprising administering to the individual the modified ASO of any one of claims 1 to 26, thereby treating or ameliorating a FOXG1 disease in the individual.

42. The method of claim 41, wherein the individual is a human.

43. The method of claim 42, wherein the human is an unborn human.

44. The method of any one of claims 40 to 43, wherein the individual comprises a mutated FOXG1 gene.

45. The method of any one of claims 40 to 43, wherein the FOXG1 disease or disorder is FOXG1 syndrome.
